(19) United States
(12) Patent Application Publication
(10) Pub. No.: US 2004/0248775 A1
(43) Pub. Date: Dec. 9, 2004

(54) Methods for screening inhibitors of apoptosis

(75) Inventors: Bradley Alton Ozenberger, Newtown, PA (US); Sanford Jay Silverman, Roosevelt, NJ (US); Jack Steven Jacobsen, Ramsey, NJ (US); Feng Liu, Plainsboro, NJ (US); Saule Naureckiene, Old Bridge, NJ (US)

Correspondence Address: WYETH, PATENT LAW GROUP, 5 GIRALDA FARMS, MADISON, NJ, 07940, US

(73) Assignee: Wyeth, Madison, NJ (US)

(21) Appl. No.: 10/860,761

(22) Filed: Jun. 3, 2004

Related U.S. Application Data
(60) Provisional application No. 60/476,269, filed on Jun. 5, 2003.

(51) Int. Cl. C12Q 1/37; A61K 38/17

(52) U.S. Cl. 514/2; 435/23

(57) ABSTRACT

The present invention addresses a need in the art for methods of identifying apoptotic proteins and methods for screening compounds which inhibit an apoptotic protein. More particularly, in certain embodiments, the invention relates to increased expression levels of a NALP1 gene and/or a NALP5 gene following neuron injury. In other embodiments, the present invention demonstrates that the recombinant expression of NALP1 and/or NALP5 polynucleotides stimulates apoptosis in cultured neurons, HeLa cells and NIH-3T3 cells. In yet other embodiments, the invention relates to mutations in the nucleotide binding sequence (NBS) of a NALP1 or a NALP5 polypeptide, wherein these NBS mutations inhibit purine nucleotide binding and reduce caspase activation.

This application claims the benefit under 35 U.S.C. §119(e) to U.S. provisional application No. 60/476,269, filed Jun. 5, 2003, which is hereby incorporated by reference in its entirety.

### FIELD OF THE INVENTION

The present invention relates generally to the fields of neuroscience and apoptosis. More particularly, in certain embodiments the invention relates to the induced expression of NALP1 and NALP5 genes following neuronal injury and methods for assaying compounds which inhibit NALP1 and/or NALP5 polypeptide activity. In other embodiments, the invention is directed to mutations in the nucleotide binding sequence of a NALP1 polypeptide or a NALP5 polypeptide, wherein these mutations reduce or inhibit caspase activation.

### BACKGROUND OF THE INVENTION

Apoptosis (programmed cell death) is the genetically determined cell suicide program resulting in distinct biochemical and morphological features. Alterations in the ability of a cell to initiate and/or execute the proper apoptotic signaling cascade have been implicated in many diseases such as cancer, autoimmune diseases, viral infections and neurodegenerative disorders (Thompson, 1995). Identifying the key mediators of apoptosis and understanding the molecular mechanisms of programmed cell death is critical to understanding these diseases.

Death domain (DD) folds have been cataloged by sequence comparisons of numerous proteins involved in apoptotic mechanisms. The involvement of death domains in protein/protein interactions was first described in death receptor signalling, characterized by multiple protein interactions. For example, the receptor-adaptor-effector complex of Fas-FAD-pro-caspase-8 requires homotypic associations between death domains or death effector domains (DED). Although pro-caspase-8 has a DED regulatory domain, caspases more commonly possess the structurally related caspase-recruitment domain (CARD). Other than caspase-9 regulation by APAF-1 (apoptosis protease-activating factor-1), which requires a physical association between the proteins mediated by their respective CARDs (Acehan et al., 2002), regulation of other caspases possessing CARD pro-domains is poorly understood.

Recently, individual components of an inflammasome were found to include caspase-1, caspase-5, the ASC adaptor protein, and NALP1 (Martinon et al., 2002). This complex includes CARD/CARD associations and homotypic interactions between another death domain fold termed the Pyrin (Py) motif, providing a regulatory scaffold for the generation of the inflammatory cytokine IL-1 from its precursor by the caspase-1 protease. The amino-terminal Py motif followed by a nucleotide binding sequence (NBS) is the identifying feature of NALP protein family (Tschopp et al., 2003). NALP1 was first identified as a CARD-containing protein (Chu et al., 2001; Hlaing et al., 2001) and its expression is observed primarily in immune cells. NALP5, which lacks a C-terminal CARD, has been reported to be expressed only in oocytes (Tong et al., 2002). Many other proteins containing predicted CARD or Py motifs have been identified by searches of DNA databases, but the functions of most of these molecules remain to be elucidated.

Most, and perhaps all, neurodegenerative diseases have an apoptotic component (Yuan and Yankner, 2000). Apoptotic phenotypes have been observed in neurons in age-related disorders such as Alzheimer's disease (Anderson et al., 1996; LeBlanc, 1996; Troncoso et al., 1996) and Parkinson's disease (Hartmann et al., 2000), and in rodent models of acute injury such as ischemic stroke (Chen et al., 1998; Namura et al., 1998). Although the etiology and progression of neurodegeneration certainly include many interconnected biochemical and physiological events, components of internally encoded programmed cell death pathways provide attractive sites for potential therapeutic intervention.

Accordingly, there is a need in the art to identify and/or modulate the various protein/protein interactions of the apoptotic pathway(s), particularly proteins which modulate the activity of caspase proteins. A better understanding of the molecular components and their function in the apoptotic pathway(s) will provide the insight needed to develop novel molecular targets for treatment or intervention in neurodegenerative disorders.

### SUMMARY OF THE INVENTION

The present invention addresses a need in the art for methods of identifying apoptotic proteins and methods for screening compounds which inhibit an apoptotic protein. More particularly, in certain embodiments, the invention relates to increased expression levels of a NALP1 gene and/or a NALP5 gene following neuron injury. In other embodiments, the present invention demonstrates that the recombinant expression of NALP1 and/or NALP5 polynucleotides stimulates apoptosis in cultured neurons, HeLa cells and NIH-3T3 cells. In yet other embodiments, the invention relates to mutations in the nucleotide binding sequence (NBS) of a NALP1 or a NALP5 polypeptide, wherein these NBS mutations inhibit purine nucleotide binding and reduce caspase activation.

Thus, in certain embodiments, the invention is directed to a method for screening compounds which inhibit NALP polypeptide activity comprising the steps of (a) providing a host cell comprising a polynucleotide expressing a NALP1 polypeptide; (b) contacting the cell with a test compound and (c) assaying caspase activity, wherein a decrease in caspase activity indicates the test compound inhibits NALP1 activity. In certain embodiments, the host cell in step (a) further comprises a polynucleotide expressing a NALP5 polypeptide. In other embodiments, the host cell is a mammalian cell. In one preferred embodiment, the mammalian cell is a HeLa cell or NIH-3T3 cell. In yet another preferred embodiment, the mammalian cell is a neuronal cell, wherein the neuronal cell is preferably a cerebellar granule neuron (CGN), a cortical neuron or a hippocampus neuron. In still other embodiments, the NALP1 polypeptide is a fusion polypeptide. In certain embodiments, the fusion polypeptide comprises an epitope tag. In a preferred embodiment, the NALP1 fusion polypeptide is a NALP1-myc-His fusion, wherein the myc-His polypeptide is at the carboxy terminus of the NALP1 polypeptide. In yet other embodiments, the NALP5 polypeptide is a fusion polypeptide. In certain embodiments, the fusion polypeptide comprises an epitope tag. In a preferred embodiment, the NALP5 fusion polypeptide is a NALP5-myc-His fusion polypeptide, wherein the myc-His polypeptide is at the carboxy terminus of the NALP5 polypeptide. In yet another preferred embodiment, assaying caspase activity comprises detecting a fluorescent caspase-3 substrate. In certain embodiments, the caspase-3 substrate is a fluorescent sulforhodamine-DEVD-FMK. In still other embodiments, the test compound is selected from the group consisting of an organic molecule, a polypeptide, a peptide fragment, a peptide mimetic, an antisense RNA and a small interference RNA. In one embodiment, the organic molecule is a nucleotide analogue, wherein the nucleotide analogue is a purine. In yet other embodiments, the polynucleotide encoding the NALP1 polypeptide comprises a nucleic acid sequence of SEQ ID NO:1. In a preferred embodiment, the polynucleotide is comprised within a mammalian expression vector. In certain embodiments, the vector is a plasmid. In certain other embodiments, the plasmid is selected from the group consisting of pcDNA3.1, pEGFP and pCMV. In yet other embodiments, the polynucleotide is operatively linked to a promoter selected from the group consisting of CMV, ADH, TRE, LTR, TK and SV40. In still other embodiments, the polynucleotide encoding the NALP5 polypeptide comprises a nucleic acid sequence of SEQ ID NO:3. In a preferred embodiment, the polynucleotide is comprised within a mammalian expression vector In one embodiment, the vector is a plasmid. In certain embodiments, the plasmid is selected from the group consisting of pcDNA3.1, pEGFP and pCMV. In other embodiments, the polynucleotide is operatively linked to a promoter selected from the group consisting of CMV, ADH, TRE, LTR, TK and SV40.

In certain other embodiments, the invention is directed to a method for screening compounds which inhibit NALP polypeptide activity comprising the steps of (a) providing a host cell comprising a polynucleotide expressing a NALP5 polypeptide; (b) contacting the cell with a test compound and (c) assaying caspase activity, wherein a decrease in caspase activity indicates the test compound inhibits NALP5 activity. In certain embodiments, the host cell in step (a) further comprises a polynucleotide expressing a NALP1 polypeptide. In other embodiments, the host cell is a mammalian cell. In one preferred embodiment, the mammalian cell is a HeLa cell or NIH-3T3 cell. In yet another preferred embodiment, the mammalian cell is a neuronal cell, wherein the neuronal cell is preferably a cerebellar granule neuron (CGN), a cortical neuron or a hippocampus neuron. In still other embodiments, the NALP1 polypeptide is a fusion polypeptide. In certain embodiments, fusion polypeptide comprises an epitope tag. In a preferred embodiment, the NALP1 fusion polypeptide is a NALP1-myc-His fusion, wherein the myc-His polypeptide is at the carboxy terminus of the NALP1 polypeptide. In yet other embodiments, the NALP5 polypeptide is a fusion polypeptide. In certain embodiments, the fusion polypeptide comprises an epitope tag. In a preferred embodiment, the NALP5 fusion polypeptide is a NALP5-myc-His fusion polypeptide, wherein the myc-His polypeptide is at the carboxy terminus of the NALP5 polypeptide. In yet another preferred embodiment, assaying caspase activity comprises detecting a fluorescent caspase-3 substrate. In certain embodiments, the caspase-3 substrate is a fluorescent sulforhodamine-DEVD-FMK. In still other embodiments, the test compound is selected from the group consisting of an organic molecule, a polypeptide, a peptide fragment, a peptide mimetic, an antisense RNA and a small interference RNA. In one embodiment, the organic molecule is a nucleotide analogue, wherein the nucleotide analogue is preferably a purine. In yet other embodiments, the polynucleotide encoding the NALP1 polypeptide comprises a nucleic acid sequence of SEQ ID NO:1. In a preferred embodiment, the polynucleotide is comprised within a mammalian expression vector. In certain embodiments, the vector is a plasmid. In certain other embodiments, the plasmid is selected from the group consisting of pcDNA3.1, pEGFP and pCMV. In yet other embodiments, the polynucleotide is operatively linked to a promoter selected from the group consisting of CMV, ADH, TRE, LTR, TK and SV40. In still other embodiments, the polynucleotide encoding the NALP5 polypeptide comprises a nucleic acid sequence of SEQ ID NO:3. In a preferred embodiment, the polynucleotide is comprised within a mammalian expression vector In one embodiment, the vector is a plasmid. In certain embodiments, the plasmid is selected from the group consisting of pcDNA3.1, pEGFP and pCMV. In other embodiments, the polynucleotide is operatively linked to a promoter selected from the group consisting of CMV, ADH, TRE, LTR, TK and SV40.

In another embodiment, the invention is directed to a method for screening compounds which inhibit NALP polypeptide activity comprising the steps of (a) providing a host cell comprising a polynucleotide expressing a NALP1 polypeptide;

(b) contacting the cell with a test compound and (c) detecting cell morphology; wherein no change in cell morphology indicates the compound inhibits NALP1 activity. In certain embodiments, the host cell in step (a) further comprises a polynucleotide expressing a NALP5 polypeptide. In other embodiments, the host cell is a mammalian cell. In one preferred embodiment, the mammalian cell is a HeLa cell or NIH-3T3 cell. In yet another preferred embodiment, the mammalian cell is a neuronal cell, wherein the neuronal cell is preferably a cerebellar granule neuron (CGN), a cortical neuron or a hippocampus neuron. In still other embodiments, the NALP1 polypeptide is a fusion polypeptide. In a preferred embodiment, the NALP1 fusion polypeptide is a NALP1-EGFP fusion, wherein the EGFP polypeptide is at the carboxy terminus of the NALP1 polypeptide. In yet other embodiments, the NALP5 polypeptide is a fusion polypeptide. In a preferred embodiment, the NALP5 fusion polypeptide is a NALP5-EGFP fusion polypeptide, wherein the EGFP polypeptide is at the carboxy terminus of the NALP5 polypeptide. In yet another preferred embodiment, assaying caspase activity comprises detecting a fluorescent caspase-3 substrate. In certain embodiments, the caspase-3 substrate is a fluorescent sulforhodamine-DEVD-FMK. In still other embodiments, the test compound is selected from the group consisting of an organic molecule, a polypeptide, a peptide fragment, a peptide mimetic, an antisense RNA and a small interference RNA. In one embodiment, the organic molecule is a nucleotide analogue, wherein the nucleotide analogue is preferably a purine. In yet other embodiments, the polynucleotide encoding the NALP1 polypeptide comprises a nucleic acid sequence of SEQ ID NO:1. In a preferred embodiment, the polynucleotide is comprised within a mammalian expression vector. In certain embodiments, the vector is a plasmid. In certain other embodiments, the plasmid is selected from the group consisting of pcDNA3.1, pEGFP and pCMV. In yet other embodiments, the polynucleotide is operatively linked to a promoter selected from the group consisting of CMV, ADH, TRE, LTR, TK and SV40. In still other embodiments, the polynucleotide encoding the NALP5 polypeptide comprises a nucleic acid sequence of SEQ ID NO:3. In a preferred embodiment, the polynucleotide is comprised within a mammalian expression vector In one embodiment, the vector is a plasmid. In certain embodiments, the plasmid is selected from the group consisting of pcDNA3.1, pEGFP and pCMV. In other embodiments, the polynucleotide is operatively linked to a promoter selected from the group consisting of CMV, ADH, TRE, LTR, TK and SV40.

In another embodiment, the invention is directed to a method for screening compounds which inhibit NALP polypeptide activity comprising the steps of (a) providing a host cell comprising a polynucleotide expressing a NALP5 polypeptide; (b) contacting the cell with a test compound and (c) detecting cell morphology; wherein no change in cell morphology indicates the compound inhibits NALP5 activity. In certain embodiments, the host cell in step (a) further comprises a polynucleotide expressing a NALP1 polypeptide. In other embodiments, the host cell is a mammalian cell. In one preferred embodiment, the mammalian cell is a HeLa cell or NIH-3T3 cell. In yet another preferred embodiment, the mammalian cell is a neuronal cell, wherein the neuronal cell is preferably a cerebellar granule neuron (CGN), a cortical neuron or a hippocampus neuron. In still other embodiments, the NALP1 polypeptide is a fusion polypeptide. In a preferred embodiment, the NALP1 fusion polypeptide is a NALP1-EGFP fusion, wherein the EGFP polypeptide is at the carboxy terminus of the NALP1 polypeptide. In yet other embodiments, the NALP5 polypeptide is a fusion polypeptide. In a preferred embodiment, the NALP5 fusion polypeptide is a NALP5-EGFP fusion polypeptide, wherein the EGFP polypeptide is at the carboxy terminus of the NALP5 polypeptide. In yet another preferred embodiment, assaying caspase activity comprises detecting a fluorescent caspase-3 substrate. In certain embodiments, the caspase-3 substrate is a fluorescent sulforhodamine-DEVD-FMK. In still other embodiments, the test compound is selected from the group consisting of an organic molecule, a polypeptide, a peptide fragment, a peptide mimetic, an antisense RNA and a small interference RNA. In one embodiment, the organic molecule is a nucleotide analogue, wherein the nucleotide analogue is preferably a purine. In yet other embodiments, the polynucleotide encoding the NALP1 polypeptide comprises a nucleic acid sequence of SEQ ID NO:1. In a preferred embodiment, the polynucleotide is comprised within a mammalian expression vector. In certain embodiments, the vector is a plasmid. In certain other embodiments, the plasmid is selected from the group consisting of pcDNA3.1, pEGFP and pCMV. In yet other embodiments, the polynucleotide is operatively linked to a promoter selected from the group consisting of CMV, ADH, TRE, LTR, TK and SV40. In still other embodiments, the polynucleotide encoding the NALP5 polypeptide comprises a nucleic acid sequence of SEQ ID NO:3. In a preferred embodiment, the polynucleotide is comprised within a mammalian expression vector In one embodiment, the vector is a plasmid. In certain embodiments, the plasmid is selected from the group consisting of pcDNA3.1, pEGFP and pCMV. Other embodiments, the polynucleotide is operatively linked to a promoter selected from the group consisting of CMV, ADH, TRE, LTR, TK and SV40.

In another embodiment, the invention is directed to a method for screening compounds which inhibit NALP polypeptide activity comprising the steps of (a) providing a host cell comprising a polynucleotide expressing a NALP1 polypeptide; (b) contacting the cell with a test compound and (c) detecting cell nuclear morphology; wherein no change in nuclear morphology indicates the compound inhibits NALP1 activity. In one embodiment, the host cell in step (a) further comprises a polynucleotide expressing a NALP5 polypeptide.

In still another embodiment, the invention is directed to a method for screening compounds which inhibit NALP polypeptide activity comprising the steps of (a) providing a host cell comprising a polynucleotide expressing a NALP5 polypeptide; (b) contacting the cell with a test compound and (c) detecting cell morphology; wherein no change in nuclear morphology indicates the compound inhibits NALP5 activity. In one embodiment, the host cell in step (a) further comprises a polynucleotide expressing a NALP1 polypeptide.

In certain other embodiments the invention is directed to a method for screening compounds which inhibit NALP polypeptide activity comprising the steps of (a) providing a host cell comprising a polynucleotide expressing a NALP1 polypeptide; (b) contacting the cell with a test compound and (c) detecting cell viability; wherein cell viability indicates the compound inhibits NALP1 activity. In one embodiment, the host cell in step (a) further comprises a polynucleotide expressing a NALP5 polypeptide.

In certain other embodiments, the invention is directed to a method for screening compounds which inhibit NALP polypeptide activity comprising the steps of (a) providing a host cell comprising a polynucleotide expressing a NALP5 polypeptide; (b) contacting the cell with a test compound and (c) detecting cell viability; wherein cell viability indicates the compound inhibits NALP5 activity. In one embodiment, the host cell in step (a) further comprises a polynucleotide expressing a NALP1 polypeptide.

In still further embodiments, the invention is directed to a method for screening compounds which inhibit apoptosis in a mammalian cell comprising the steps of (a) providing a host cell comprising a polynucleotide expressing a NALP1 polypeptide; (b) contacting the cell with a test compound and (c) assaying caspase activity, wherein a decrease in caspase activity indicates the test compound inhibits NALP1 activity.

In another embodiment, the invention is directed to a method for screening compounds which inhibit apoptosis in a mammalian cell comprising the steps of (a) providing a host cell comprising a polynucleotide expressing a NALP1 polypeptide; (b) contacting the cell with a test compound and (c) detecting cell morphology; wherein no change in cell morphology indicates the compound inhibits NALP1 activity.

In certain other embodiments, the invention is directed to a method for screening compounds which inhibit apoptosis in a mammalian cell comprising the steps of (a) providing a host cell comprising a polynucleotide expressing a NALP1 polypeptide; (b) contacting the cell with a test compound and (c) detecting nuclear morphology; wherein no change in nuclear morphology indicates the compound inhibits NALP1 activity.

In yet another embodiment, the invention is directed to a method for screening compounds which inhibit apoptosis in a mammalian cell comprising the steps of (a) providing a host cell comprising a polynucleotide expressing a NALP1 polypeptide; (b) contacting the cell with a test compound and (c) detecting cell viability; wherein cell viability indicates the compound inhibits NALP1 activity.

In certain other embodiments, the invention is directed to a method for detecting neuron damage in a mammalian subject comprising the steps of (a) obtaining a biological sample from the subject; (b) contacting the sample with a polynucleotide probe complementary to a NAPL1 mRNA or a NALP5 mRNA; (c) measuring the amount of probe bound to the mRNA and (d) comparing the amount in step (c) with NALP1 mRNA or NALP5 mRNA in mammalian samples obtained from a statistically significant population lacking neuron damage, wherein higher NALP1 or NALP5 levels in the subject indicates neuron damage.

In other embodiments, the invention is directed to a method for detecting neuron damage in a mammalian subject comprising the steps of (a) obtaining a biological sample from the subject; (b) contacting the sample with a polynucleotide probe complementary to a NAPL1 mRNA and a polynucleotide probe complementary to a NALP5 mRNA; (c) measuring the amount of each probe bound to the mRNA and (d) comparing the amount in step (c) with NALP1 mRNA and NALP5 mRNA in mammalian samples obtained from a statistically significant population lacking neuron damage, wherein higher NALP1 or NALP5 levels in the subject indicates neuron damage. In certain embodiments, the probe complementary to the NALP1 mRNA comprises a nucleotide sequence which hybridizes under high stringency hybridization conditions with a polynucleotide comprising the nucleotide sequence of SEQ ID NO:1. In still other embodiments, the probe complementary to the NALP5 mRNA comprises a nucleotide sequence which hybridizes under high stringency hybridization conditions with a polynucleotide comprising the nucleotide sequence of SEQ ID NO:3. In still other embodiments, the biological sample is selected from the group consisting of blood plasma, serum, erythrocytes, leukocytes, platelets, lymphocytes, macrophages, fibroblast cells, mast cells, fat cells, epithelial cells, nerve cells, glial cells, Schwann cells, progenitor stem cells, a cerebrospinal fluid (CSF), saliva, a skin biopsy, a brain biopsy and a buccal biopsy. In yet another embodiment, the polynucleotide probe is labeled with a radioactive isotope or a fluorophore.

In another embodiment, the invention is directed to a method for measuring the expression levels of a NALP1 gene and NALP5 gene in a rat neuron comprising the steps of (a) obtaining a cultured rat neuron cell; (b) isolating the total RNA from step (a); (c) generating a NALP1 cDNA and a NALP5 cDNA from the RNA of step (b) by PCR using a 5′ NALP1 PCR primer comprising a nucleic acid sequence of SEQ ID NO:23, a 3′ NALP1 PCR primer comprising a nucleic acid sequence of SEQ ID NO:24; a 5′ NALP5 PCR primer comprising a nucleic acid sequence of SEQ ID NO:23 and a 3′ NALP5 PCR primer comprising a nucleic acid sequence of SEQ ID NO:24 and (d) detecting the amount of the cDNA in step (c). In preferred embodiments, the neuron cell is a CGN, a cortical neuron or a hippocampus neuron. In another embodiment, the cDNA comprises a radioactive dNTP.

In certain other embodiments, the invention is directed to a method for assaying neuron damage or injury in a rat neuron cell comprising the steps of (a) obtaining a cultured rat neuron cell; (b) injuring the cell by transfer to a culture medium having no serum and a reduced K⁺ concentration of about 5 mM; (c) isolating the total RNA from step (b); (d) generating a NALP1 cDNA and a NALP5 cDNA from the RNA of step (c) by PCR using a 5′ NALP1 PCR primer comprising a nucleic acid sequence of SEQ ID NO:23, a 3′ NALP1 PCR primer comprising a nucleic acid sequence of SEQ ID NO:24; a 5′ NALP5 PCR primer comprising a nucleic acid sequence of SEQ ID NO:23 and a 3′ NALP5 PCR primer comprising a nucleic acid sequence of SEQ ID NO:24 and (e) detecting the amount of the cDNA in step (d), wherein an increase in either NALP1 or NALP5 cDNA in step (e), relative to a non-injured neuron control, indicates neuron injury or damage.

In yet another embodiment, the invention is directed to a method for monitoring the kinetics of neuron injury comprising the steps of (a) subjecting a population of adults rats to transient middle cerebral artery occlusion (MCAO) for about 1 hour and immediately reperfusing; (b) obtaining at a desired kinetic time point a rat from step (a), wherein cortex tissue from the rat is dissected and frozen; (c) repeating step (b) for each desired time point; (d) isolating the total RNA from the tissue in each time point; (e) generating a NALP1 cDNA and a NALP5 cDNA from the RNA of step (d) by PCR using a 5′ NALP1 PCR. primer comprising a nucleic acid sequence of SEQ ID NO:23, a 3′ NALP1 PCR primer comprising a nucleic acid sequence of SEQ ID NO:24; a 5′ NALP5 PCR primer comprising a nucleic acid sequence of SEQ ID NO:23 and a 3′ NALP5 PCR primer comprising a nucleic acid sequence of SEQ ID NO:24 and (f) detecting the amount of the cDNA in step (e).

In another embodiment, the invention is directed to a method for screening compounds which inhibit the expression of a NALP1 polypeptide comprising the steps of (a) providing a host cell comprising a polynucleotide expressing a NALP1 polypeptide; (b) contacting the cell with a test compound and (c) assaying NALP1 gene expression, wherein a decrease in NALP1 gene expression indicates the test compound inhibits the NALP1 apoptosis pathway.

In still another embodiment, the invention is directed to a method for screening compounds which inhibit the expression of a NALP5 polypeptide comprising the steps of (a) providing a host cell comprising a polynucleotide expressing a NALP5 polypeptide; (b) contacting the cell with a test compound and (c) assaying NALP5 gene expression, wherein a decrease in NALP5 gene expression indicates the test compound inhibits the NALP5 apoptosis pathway.

In other embodiments, the invention is directed to an antisense RNA molecule which inhibits the expression of a polynucleotide encoding a NALP1 polypeptide comprising an amino acid sequence of SEQ ID NO:2. In certain embodiments, the RNA molecule is antisense to a polynucleotide having a nucleotide sequence of SEQ ID NO:1 or a degenerate variant thereof. In a preferred embodiment, the RNA molecule comprises a nucleotide sequence of SEQ ID NO:5.

In another embodiment, the invention is directed to an antisense RNA molecule which inhibits the expression of a polynucleotide encoding a NALP5 polypeptide comprising an amino acid sequence of SEQ ID NO:4. In certain embodiments, the RNA molecule is antisense to a polynucleotide having a nucleotide sequence of SEQ ID NO:3 or a degenerate variant thereof.

In another embodiment, the invention is directed to a method for inhibiting apoptosis in a cell comprising administering to the cell an expression construct comprising an RNA molecule antisense to SEQ ID NO:1 or SEQ ID NO:3.

In yet other embodiments, the invention is directed to a polynucleotide encoding a mutated NALP1 polypeptide comprising an amino acid sequence of SEQ ID NO:2, wherein the glycine amino acid at position 339 of SEQ ID NO:2 is mutated to a glutamate amino acid.

In still other embodiments, the invention is directed to a polynucleotide encoding a mutated NALP1 polypeptide comprising an amino acid sequence of SEQ ID NO:2, wherein the lysine amino acid at position 340 of SEQ ID NO:2 is mutated to an alanine amino acid.

In another embodiment, the invention is directed to a polynucleotide encoding a mutated NALP1 polypeptide comprising an amino acid sequence of SEQ ID NO:2, wherein the glycine amino acid at position 339 of SEQ ID NO:2 is mutated to a glutamate amino acid and the lysine amino acid at position 340 of SEQ ID NO:2 is mutated to an alanine amino acid.

In certain other embodiments, the invention is directed to a polynucleotide encoding a NALP1 polypeptide of SEQ ID NO:2, wherein the amino acid sequence of SEQ ID NO:2 comprises a mutation in the nucleotide binding sequence (NBS), wherein the NBS comprises amino acid 328 through amino acid 637 of SEQ ID NO:2. In one particular embodiment, a mutation in the NBS is further defined as a mutation in the Mg²⁺ binding sequence of SEQ ID NO:2 comprising amino acid 392 through amino acid 415. In a preferred embodiment, a mutation in the Mg²⁺ binding sequence of SEQ ID NO:2 is a mutation at an amino acid residue selected from the group consisting of glutamate 403 (Glu 403), aspartate 410 (Asp 410), aspartate 413 (Asp 413) and glutamate 414 (Glu 414). In preferred embodiments, a NALP1 polypeptide with a mutation in the NBS does not bind a purine nucleotide, most preferably the NALP1 polypeptide does not bind dATP.

In certain other embodiments, the invention is directed to a polypeptide comprising an amino acid sequence of SEQ ID NO:2, wherein the glycine amino acid at position 339 of SEQ ID NO:2 is mutated to a glutamate amino acid.

In another embodiment, the invention is directed to a polypeptide comprising an amino acid sequence of SEQ ID NO:2, wherein the lysine amino acid at position 340 of SEQ ID NO:2 is mutated to an alanine amino acid.

In yet another embodiment, the invention is directed to a polypeptide comprising an amino acid sequence of SEQ ID NO:2, wherein the glycine amino acid at position 339 of SEQ ID NO:2 is mutated to a glutamate amino acid and the lysine at amino acid position 340 of SEQ ID NO: is mutated to an alanine amino acid.

In certain other embodiments, the invention is directed to a polypeptide comprising an amino acid sequence of SEQ ID NO:2, wherein the amino acid sequence of SEQ ID NO:2 comprises a mutation in the NBS, wherein the NBS comprises amino acid 328 to amino acid 637 of SEQ ID NO:2. In one particular embodiment, a mutation NBS is further defined as a mutation in the Mg²⁺ binding sequence of SEQ ID NO:2 comprising amino acid 392 through amino acid 415. In a preferred embodiment, a mutation in the Mg²⁺ binding sequence of SEQ ID NO:2 is a mutation at am amino acid residue selected from the group consisting of Glu 403, Asp 410, Asp 413 and Glu 414. In preferred embodiments, a NALP1 polypeptide with a NBS mutation does not bind a purine nucleotide, most preferably NALP1 does not bind dATP.

In other embodiments, the invention is directed to a method for screening compounds which activate a NALP1 polypeptide comprising the steps of (a) providing a host cell comprising a polynucleotide encoding a NALP1 polypeptide having a mutation in the NBS; (b) contacting the cell with a test compound and (c) assaying NALP1 activity, wherein an increase in NALP1 activity indicates the compound activates the polypeptide. In a preferred embodiment, the test compound is a nucleotide analogue of GTP, dGTP, ATP or dATP.

In still other embodiments, the invention is directed to a polynucleotide encoding a NALP5 polypeptide of SEQ ID NO:4, wherein the amino acid sequence of SEQ ID NO:4 comprises a mutation in the nucleotide binding sequence (NBS) from amino acid 191 to amino acid 510. In certain embodiments, a mutation in the NBS is further defined as a mutation in the Mg²⁺ binding sequence of SEQ ID NO:4, wherein the Mg²⁺ binding sequence comprises amino acid 357 through amino acid 367 of SEQ ID NO:4. In a preferred embodiment, a mutation in the Mg²⁺ binding sequence of SEQ ID NO:4 is a mutation at an amino acid residue selected from the group consisting of Asp 362, Asp 365 and Asp 366. In a preferred embodiment, the NALP5 polypeptide does not bind a purine nucleotide, most preferably the NALP5 polypeptide does not bind the purine nucleotide dATP.

In certain other embodiments, the invention is directed to a polypeptide comprising an amino acid sequence of SEQ ID NO:4, wherein the amino acid sequence of SEQ ID NO:4 comprises a mutation in the NBS from amino acid 191 to amino acid 510. In another embodiment, a mutation in the NBS is further defined as a mutation in the Mg²⁺ binding sequence of SEQ ID NO:2, wherein the Mg²⁺ binding sequence comprises amino acid 357 through amino acid 367 of SEQ ID NO:4. In a preferred embodiment, a mutation in the Mg²⁺ binding sequence of SEQ ID NO:2 is a mutation at an amino acid residue selected from the group consisting of Asp 362, Asp 365 and Asp 366. In certain preferred embodiments, the NALP5 polypeptide does not bind a purine nucleotide, most preferably the NALP5 polypeptide does not bind the purine nucleotide dATP.

In still another embodiment, the invention is directed to a method for screening compounds which activate a NALP5 polypeptide comprising the steps of: (a) providing a host cell comprising a polynucleotide encoding a NALP5 polypeptide having a mutation in the NBS; (b) contacting the cell with a test compound and (c) assaying NALP5 activity, wherein an increase in NALP5 activity indicates the compound activates the polypeptide. In a preferred embodiment, the test compound is a nucleotide analogue of dGTP, GTP, ATP or dATP.

In certain preferred embodiments, the invention provides a pharmaceutical composition comprising a compound identified according to methods set forth in the present invention.

Other features and advantages of the invention will be apparent from the following detailed description, from the preferred embodiments thereof, and from the claims.

### BRIEF DESCRIPTION OF THE DRAWINGS

FIG. 1 is a schematic representation of the putative domains in the ASC protein, the NALP1 protein, the NALP5 protein, the CARD8 protein and the CARD9 protein. The scale is approximate and the numbers indicate amino acids contained in the most common human variant of each protein. Py, pyrin death domain motif; CARD, caspase recruitment domain; NBS, nucleotide binding sequence; LRR, leucine-rich repeats; dashed line, region of 50% identify between NALP1 and CARD8.

FIG. 2 is a gene expression analysis in cultured neurons. Images show PCR amplicons visualized after electrophoresis through an agarose gel containing ethidium bromide, and ultraviolet illumination. First strand cDNA was generated from cultured neuron samples and used as template for PCRs as described in Example 1. Primer pairs were specific for the indicated gene, as confirmed by determination of the DNA sequence of each amplicon. Lane 1, no template; lane 2, cortical neurons; lane 3, untreated cerebellar granule neurons; lane 4, cerebellar granule neurons injured by withdrawal of serum and K⁺; lane 5, rat genomic DNA.

FIG. 3 is a gene expression analysis in cortical tissue following transient focal ischemia and reperfusion. Images show PCR amplicons as described in FIG. 2. Paired samples were generated from cortex of the control (contralateral; C) hemisphere or the ischemic (ipsilateral; I) hemisphere of the same animal. The left-most samples were derived from a sham-operated animal. Other samples were isolated at the indicated times after 1 hour transient ischemia.

FIG. 4 demonstrates that the expression of recombinant NALP1 or NALP5 stimulates apoptosis. FIG. 4A, HeLa cells expressing tagged NALP1 or NALP5 proteins were stained with Hoechst dye to reveal nuclear morphology and with the fluorogenic DEVDase substrate. Transfected cells were scored manually for pyknotic nuclear morphology or DEVDase activity by fluorescence microscopy. Values are means with standard deviation (SD) of duplicate samples of a single representative experiment. Each pNALP1 or pNALP5 value, derived from >200 transfected cells, was compared to pEGFP using a Yates G-test method for categorical data, and found to be highly significant (P<0.01). FIG. 4B, Rat cerebellar granule neurons in culture were transfected and investigated for apoptotic phenotype as described above. Data from a representative experiment are shown. Values represent means with SD of duplicate samples. NALP1-EGFP and NALP5-EGFP effects versus vector control were highly significant (P<0.01).

FIG. 5 demonstrates that knockdown of native NALP1 protects HeLa cells from apoptotic insult. FIG. 5A, RNA samples were isolated from normal HeLa cells and transcribed into first-strand cDNA. PCR amplifications were performed for both NALP1 and GAPDH, a gene of moderate expression levels, with a 10-fold dilution series extrapolated to the indicated RNA amounts. The expression level of NALP1 is substantially (˜10 times) less than that of GAPDH. HeLa cells were transfected with control (scrambled) siRNA or siRNA directed to NALP1. RNA was isolated 48 hours after transfection. RT-PCR amplifications were performed using 200 ng RNA for NALP1 or 100 ng RNA for GAPDH. NALP1 siRNA substantially reduced NALP1 mRNA levels with no effect on GAPDH (right panels). FIG. 5B, samples prepared in parallel with those described above were treated with 1 μM etoposide for an additional 24 hours. Cell lysates were generated and investigated for caspase-3 activation by immunoblot. No activated caspase-3 was detectable in the absence of etoposide (no injury). The same blots were probed with anti-actin antibody to monitor protein loading. Similar results were obtained from three independent experiments.

FIG. 6 shows that NALP1 binds to dATP through its nucleotide binding sequence (NBS). An expression plasmid for His6-NALP1 was engineered. This plasmid was altered by oligonucleotide-directed mutagenesis to express His6-NALP1 with two amino acid substitutions (G339E, K340A) in the predicted P-loop of the NBS. COS-7 cells were transfected with expression plasmids for wild-type or NBS mutant His6-NALP1, or vector alone. His6-tagged proteins were purified from lysates by Ni⁺² affinity chromatography. FIG. 6A, purified NALP1 proteins eluted and analyzed by SDS/PAGE followed by immunoblotting with anti-His6 antibody. Lane 1, vector; lane 2, His6-NALP1-wildtype (wt); lane 3, His6-NALP1-mutant (mut). FIG. 6B, proteins were immobilized on a Ni⁺² flashplate and investigated for binding of dATPα³⁵S. Specific binding was determined by measuring total binding and subtracting radionucleotide bound in presence of cold dATP in large excess. Representative data are shown as means±SD. Statistical analyses (Student t-test) indicated that the effect of mutant versus wild-type is significant (p<0.01), and the effect of mutant versus vector is not significant (p>0.05). Several (N=4) independent experiments produced the same results.

FIG. 7 demonstrates that mutation of the NALP1 NBS attenuates pro-apoptotic activity. HeLa cells transiently expressing wild-type (wt) or NBS mutant (mut) NALP1-EGFP proteins or EGFP alone were treated with sulforhodamine-DEVD-FMK to detect active caspase-3. Cells were scored manually for protein expression and DEVDase activity by fluorescence microscopy. The quantitation of caspase-3 activity was expressed as the percentage of transfected cells. Values are means with SD from duplicate samples, and are representative of multiple (N=4) experiments. Cell lysates from parallel samples were examined by immunoblot using an anti-GFP antibody to confirm that wild-type and NBS mutant NALP1-EGFP proteins were expressed at similar levels (inset).

FIG. 8 shows the addition of dATP in vitro stimulates NALP1-dependent caspase activation. HeLa cells transiently expressing wild-type or NBS mutant NALP1-EGFP proteins or EGFP alone were homogenized and soluble extract prepared 24 hours after plasmid transfection. Extracts were aliquoted and incubated with or without the addition of dATP (2 mM) and MgCl₂ (2 mM) followed by either DEVDase activity measurements using synthetic fluorogenic substrate (FIG. 8A) or direct investigation of caspase-3 activation by immunoblot (FIG. 8B). Triplicate samples were measured with or without addition of peptide inhibitor. Fluorescence measurements were collected and values in the presence of inhibitor were subtracted from values without inhibitor to obtain specific DEVDase activity. Values were normalized to the EGFP control reaction in the absence of added dATP. Data from six independent experiments are expressed as means±SD, with statistical significance determined by Student t-test (#, p<0.05, compared to vector alone without dATP; *, p<0.05, compared to wild-type NALP1 without dATP). Parallel samples were analyzed by SDS/PAGE and immunoblot with anti-GFP and anti-active caspase-3 antibodies.

### DETAILED DESCRIPTION OF THE INVENTION

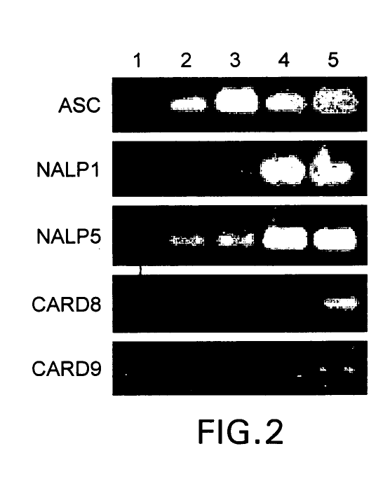

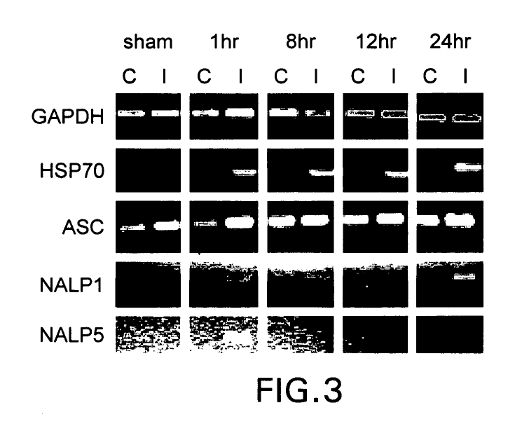

The present invention addresses a need in the art for methods of assaying apoptotic proteins and methods for screening compounds which inhibit apoptotic proteins. More particularly, in certain embodiments, the invention relates to increased expression levels of the NALP1 gene and the NALP5 gene following neuron injury. NALP1 expression is observed primarily in immune cells, whereas NALP5 has been reported to be expressed only in oocytes. Surprisingly however, the expression of both NALP1 and NALP5 was substantially elevated following injury in neuronal culture (see, Example 2 and FIG. 2) or after transient cerebral artery occlusion and reperfusion (see, Example 3 and FIG. 3). These findings indicate that the NALP1 and NALP5 gene products function in injured neurons, in addition to their activities in inflammation or early embryonic development in other cell types. In other embodiments, the invention demonstrates that the recombinant expression of NALP1 and/or NALP5 stimulate apoptosis in cultured neurons, HeLa cells and NIH-3T3 cells (Example 4).

It was also observed in the present invention that caspase-3 activation was substantially attenuated by a small interfering RNA (siRNA) complementary to NALP1 (Example 5), suggesting an important function of NALP1 in transducing an apoptosis signal. In yet other embodiments, the invention relates to mutations in the nucleotide binding sequence (NBS) of a NALP1 or a NALP5 polypeptide, wherein these NBS mutations inhibit purine nucleotide binding and reduce caspase activation. For example, a NALP1 NBS double mutant was generated comprising a glycine to glutamic acid substitution at amino acid position 339 (G339E) of SEQ ID NO:2 and a lysine to alanine substitution at amino acid position 340 (K340A) of SEQ ID NO:2, wherein the double mutation (i.e., G339E and K340A) is comprised within the P-loop motif of the NALP1 NBS (i.e., the NBS comprises amino acid residues 328 to 637 of SEQ ID NO:2). The wild-type NALP1 polypeptide showed specific binding of dATP in radionucleotide assays in vitro, whereas the NBS mutant did not, demonstrating that NALP1 binds dATP through its NBS domain (Example 6). Complementary to the in vivo data, the inclusion of dATP in HeLa cell extracts stimulated caspase activation by wild-type NALP1, but no effect was observed with NBS mutant protein (Example 7), confirming that the nucleotide binding function of NALP1 is important for its pro-apoptotic activity.

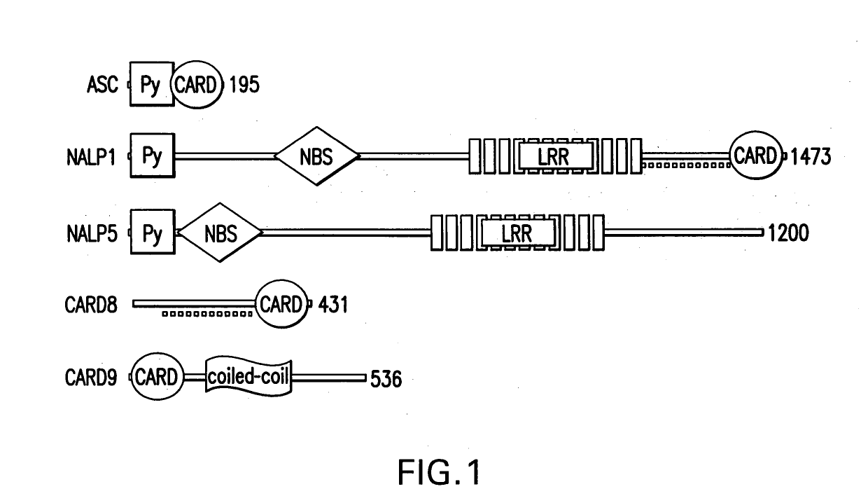

As defined hereinafter, a “NALP1” polypeptide and a “NALP5” polypeptide are members of the NALP protein (NACHT Leucine-rich repeat Protein) family and are characterized by an N-terminal Pyrin (Py) motif followed by a purine binding site (see, FIG. 1). As defined hereinafter, a “NALP1 polypeptide” of the invention comprises an N-terminal Py domain, followed by a putative NBS, a putative regulatory domain containing multiple leucine-rich repeats (LRRs) and a C-terminal caspase-recruitment domain (CARD) (FIG. 1). As defined hereinafter, a “NBS” or a “nucleotide binding sequence” of NALP1 polypeptide comprises amino acid residues 328 to 637 of SEQ ID NO:2. Similarly, as defined hereinafter, a “Mg²⁺ binding motif” of a NALP1 polypeptide comprises amino acid residues 392 to 415 of SEQ ID NO:2.

As defined hereinafter, a “NALP5 polypeptide” of the invention comprises an N-terminal Py domain, followed by a putative NBS, a putative regulatory domain containing multiple leucine-rich repeats (LRRs), but lacks the C-terminal CARD contained in NALP1 (FIG. 1). As defined hereinafter, a “NBS” or a “nucleotide binding sequence” of NALP5 polypeptide comprises amino acid residues 191 to 510 of SEQ ID NO:4. Similarly, as defined hereinafter, a “Mg²⁺ binding motif” of a NALP5 polypeptide comprises amino acid residues 357 to 367 of SEQ ID NO:4.

Additionally, the NALP1 polypeptide is also known in the art as CARD7, NAC and DEFCAP, and as such, hereinafter any reference to a “NALP1 gehe”, a “NALP1 polynucleotide” or a “NALP1 polypeptide” is meant to include CARD7, NAC and DEFCAP. The NALP5 polypeptide is also known in the art as MATER and Pyrin5, and as such, hereinafter any reference to a “NALP5 gene”, a “NALP5 polynucleotide” or a “NALP5 polypeptide” is meant to include MATER and Pyrin5.

A. Isolated Polynucleotides

In certain embodiments, the invention is directed to methods for screening compounds which inhibit NALP polypeptide activity in a host cell comprising a NALP1 and/or NALP5 polynucleotide expressing said polypeptide. In other embodiments, the invention is directed to methods for screening compounds which modulate apoptosis in a mammalian cell via inhibiting NALP polypeptide activity. In other embodiments, the invention is directed to a method for detecting neuron damage in a mammalian cell by contacting a biological sample with a polynucleotide probe complementary to a NALP1 or a NALP5 mRNA. In still other embodiments, the invention is directed to recombinantly expressed mutant NALP1 and/or NALP5 polypeptides, wherein the polynucleotide encoding the NALP1 polypeptides comprises a nucleic acid sequence of SEQ ID NO:1 and the polynucleotide encoding the NALP5 polypeptides comprises a nucleic sequence of SEQ ID NO:3.

Thus, in one aspect, the present invention provides isolated and purified polynucleotides that encode NALP1 and NALP5 polypeptides. In a preferred embodiment, a polynucleotide of the invention is a recombinant polynucleotide which encodes a human NALP1 polypeptide or a human NALP5 polypeptide. In other embodiments, a polynucleotide encoding a NALP1 polypeptide or a NALP5 polypeptide is rodent (e.g., a rat or mouse orthologue) polynucleotide. An isolated polynucleotide encoding a human NALP1 polypeptide of SEQ ID NO:2 has a nucleotide sequence shown in SEQ ID NO:1. An isolated polynucleotide encoding a human NALP5 polypeptide of SEQ ID NO:4 has a nucleotide sequence shown in SEQ ID NO:3.

The invention further encompasses polynucleotides that differ from the NALP1 nucleotide sequence of SEQ ID NO:1 or the NALP5 nucleotide sequence of SEQ ID NO:3 (e.g., an orthologue or an allelic variant). For example, due to the degeneracy of the genetic code, a NALP1 polynucleotide of the invention is any polynucleotide encoding a NALP1 polypeptide having at least about 80%, more preferably about 90% and even more preferably about 95% sequence identity to a NALP1 polypeptide of SEQ ID NO:2. Similarly, a NALP5 polynucleotide of the invention is any polynucleotide encoding a NALP5 polypeptide having at least about 80%, more preferably at least about 90% and even more preferably at least about 95% sequence identity to a NALP5 polypeptide of SEQ ID NO:4. Such nucleic acid molecules are readily identified as being able to hybridize, preferably under stringent conditions, to the nucleotide sequence shown in SEQ ID NO:1 or SEQ ID NO:3.

As used herein, the term “polynucleotide” means a sequence of nucleotides connected by phosphodiester linkages. Polynucleotides are presented herein in the direction from the 5′ to the 3′ direction. A polynucleotide of the present invention comprises from about 40 to about several hundred thousand base pairs. Preferably, a polynucleotide comprises from about 10 to about 3,000 base pairs. Preferred lengths of particular polynucleotide are set forth hereinafter.

A polynucleotide of the present invention is a deoxyribonucleic acid (DNA) molecule, a ribonucleic acid (RNA) molecule, or analogs of the DNA or RNA generated using nucleotide analogs. The nucleic acid molecule is single-stranded or double-stranded, but preferably is double-stranded DNA. Where a polynucleotide is a DNA molecule, that molecule is a plasmid DNA, a cDNA molecule or a genomic DNA molecule. Nucleotide bases are indicated herein by a single letter code: adenine (A), guanine (G), thymine (T), cytosine (C), inosine (I) and uracil (U).

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

Polynucleotides of the present invention are obtained, using standard cloning and screening techniques, from a cDNA library derived from mRNA from human cells or from genomic DNA. Polynucleotides of the invention are also synthesized using well known and commercially available techniques.

In another preferred embodiment, an isolated polynucleotide of the invention comprises a nucleic acid molecule which is a complement of the nucleotide sequence shown in SEQ ID NO:1 or SEQ ID NO:3, or a fragment of one of these nucleotide sequences. A nucleic acid molecule which is complementary to the nucleotide sequence shown in SEQ ID NO:1 or SEQ ID NO:3 is one which is sufficiently complementary to the nucleotide sequence, such that it can hybridize to the nucleotide sequence shown in SEQ ID NO:1 or SEQ ID NO:3, thereby forming a stable duplex. Examples of hybridization stringency conditions are detailed in Table 1. Moreover, the polynucleotide of the invention can comprise only a fragment of the coding region of a polynucleotide or gene, such as a fragment of SEQ ID NO:1 or SEQ ID NO:3.

When the polynucleotides of the invention are used for the recombinant production of NALP1 and/or NALP5 polypeptides, the polynucleotide includes the coding sequence for the mature polypeptide, by itself, or the coding sequence for the mature polypeptide in reading frame with other coding sequences, such as those encoding a leader or secretory sequence, a pre-, a pro- or a pre-pro-polypeptide sequence, an epitope sequence or other fusion peptide portions. For example, a marker sequence which facilitates purification of the fused polypeptide can be encoded (e.g., see Gentz et al., 1989 and Section B). The polynucleotide may also contain non-coding 5′ and 3′ sequences, such as transcribed, non-translated sequences, splicing and polyadenylation signals, ribosome binding sites and sequences that stabilize mRNA (e.g., see Section C).

In certain embodiments, the polynucleotide sequence information provided by the present invention allows for the preparation of relatively short DNA (or RNA) oligonucleotide sequences having the ability to specifically hybridize to gene sequences of the selected polynucleotides disclosed herein. In a preferred embodiment, an oligonucleotide sequence is one which is complimentary to a NALP1 or NALP5 mRNA. The term “oligonucleotide” as used herein is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence, e.g., a sequence such as that shown in SEQ ID NO:1 or SEQ ID NO:3. The ability of such nucleic acid probes to specifically hybridize to a polynucleotide encoding a NALP polypeptide lends them particular utility in a variety of embodiments. Most importantly, the probes are used in a variety of assays for detecting the presence of complementary sequences in a given sample.

In certain embodiments, it is advantageous to use oligonucleotide primers. These primers are generated in any manner, including chemical synthesis, DNA replication, reverse transcription, or a combination thereof. The sequence of such primers is designed using a polynucleotide of the present invention for use in detecting, amplifying or mutating a defined segment of a gene or polynucleotide that encodes a polypeptide from mammalian cells using polymerase chain reaction (PCR) technology.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

To provide certain advantages in accordance with the present invention, a preferred nucleic acid sequence employed for hybridization studies or assays includes probe molecules that are complementary to at least a 10 to 70 or so long nucleotide stretch of a polynucleotide that encodes a polypeptide of the invention. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. One will generally prefer to design nucleic acid molecules having gene-complementary stretches of 25 to 40 nucleotides, 55 to 70 nucleotides, or even longer where desired. Such fragments are readily prepared by, for example, directly synthesizing the fragment by chemical means, by application of nucleic acid reproduction technology, such as the PCR technology of U.S. Pat. No. 4,683,202 (incorporated by reference herein in its entirety) or by excising selected DNA fragments from recombinant plasmids containing appropriate inserts and suitable restriction enzyme sites.

Accordingly, a polynucleotide probe molecule of the invention can be used for its ability to selectively form duplex molecules with complementary stretches of the gene. Depending on the application envisioned, one will desire to employ varying conditions of hybridization to achieve a varying degree of selectivity of the probe toward the target sequence. For applications requiring a high degree of selectivity, one will typically desire to employ relatively stringent conditions to form the hybrids (see Table 1 below).

The present invention also includes polynucleotides capable of hybridizing under reduced stringency conditions, more preferably stringent conditions, and most preferably highly stringent conditions, to polynucleotides described herein. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
HYBRIDIZATION STRINGENCY CONDITIONS

| | | | Hybridization | Wash |
|---|---|---|---|---|
| Stringency | Polynucleotide | Hybrid Length | Temperature and | Temperature |
| Condition | Hybrid | (bp)¹ | Buffer^{H} | and BufferH |
| A | DNA:DNA | >50 | 65° C.; 1 × SSC -or- | 65° C.; 0.3 × SSC |
| | | | 42° C.; 1 × SSC, 50% | |
| | | | formamide | |
| B | DNA:DNA | <50 | T_{B}; 1 × SSC | T_{B}; 1 × SSC |
| C | DNA:RNA | >50 | 67° C.; 1 × SSC -or- | 67° C.; 0.3 × SSC |
| | | | 45° C.; 1 × SSC, 50% | |
| | | | formamide | |
| D | DNA:RNA | <50 | T_{D}; 1 × SSC | T_{D}; 1 × SSC |
| E | RNA:RNA | >50 | 70° C.; 1 × SSC -or- | 70° C.; 0.3 × SSC |
| | | | 50° C.; 1 × SSC, 50% | |
| | | | formamide | |
| F | RNA:RNA | <50 | T_{F}; 1 × SSC | T_{f}; 1 × SSC |
| G | DNA:DNA | >50 | 65° C.; 4 × SSC -or- | 65° C.; 1 × SSC |
| | | | 42° C.; 4 × SSC, 50% | |
| | | | formamide | |
| H | DNA:DNA | <50 | T_{H}; 4 × SSC | T_{H}; 4 × SSC |
| I | DNA:RNA | >50 | 67° C.; 4 × SSC -or- | 67° C.; 1 × SSC |
| | | | 45° C.; 4 × SSC, 50% | |
| | | | formamide | |
| J | DNA:RNA | <50 | T_{J}; 4 × SSC | T_{J}; 4 × SSC |
| K | RNA:RNA | >50 | 70° C.; 4 × SSC -or- | 67° C.; 1 × SSC |
| | | | 50° C.; 4 × SSC, 50% | |
| | | | formamide | |
| L | RNA:RNA | <50 | T_{L}; 2 × SSC | T_{L}; 2 × SSC |
| M | DNA:DNA | >50 | 50° C.; 4 × SSC -or- | 50° C.; 2 × SSC |
| | | | 40° C.; 6 × SSC, 50% | |
| | | | formamide | |
| N | DNA:DNA | <50 | T_{N}; 6 × SSC | T_{N}; 6 × SSC |
| O | DNA:RNA | >50 | 55° C.; 4 × SSC -or- | 55° C.; 2 × SSC |
| | | | 42° C.; 6 × SSC, 50% | |
| | | | formamide | |
| P | DNA:RNA | <50 | T_{P}; 6 × SSC | T_{P}; 6 × SSC |
| Q | RNA:RNA | >50 | 60° C.; 4 × SSC -or- | 60° C.; 2 × SSC |
| | | | 45° C.; 6 × SSC, 50% | |
| | | | formamide | |
| R | RNA:RNA | <50 | T_{R}; 4 × SSC | T_{R}; 4 × SSC |
| (bp)¹: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length is determined by aligning the sequences of the polynucleotides and identifying the region or regions of optimal sequence complementarity. | | | | |
| Buffer^{H}: SSPE (1 × SSPE is 0.15M NaCl, 10 mM NaH₂PO₄, and 1.25 mM EDTA, pH 7.4) can be substituted for SSC (1 × SSC is 0.15M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 | | | | |
| # and 49 base pairs in length, Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1 × SSC = 0.165 M). | | | | |

In addition to the nucleic acid molecules encoding NALP1 and NALP5 polypeptides described above, another aspect of the invention pertains to isolated nucleic acid molecules which are antisense to NALP1 or NALP5. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid is complementary to an entire NALP1 or NALP5 coding strand (e.g., SEQ ID NO:1 or SEQ ID NO:3), or to only a fragment thereof. In one embodiment, an antisense nucleic acid molecule is antisense to a “coding region” of the coding strand of a nucleotide sequence encoding a NALP polypeptide.

The term “coding region” refers to the region of the nucleotide sequence comprising codons which are translated into amino acid residues, e.g., the entire coding region of SEQ ID NO:2 or SEQ ID NO:4. In another embodiment, the antisense nucleic acid molecule is antisense to a “non-coding region” of the coding strand of a nucleotide sequence encoding a NALP polypeptide. The term “non-coding region” refers to 5′ and 3′ sequences which flank the coding region that are not translated into amino acids (i.e., also referred to as 5′ and 3′ untranslated regions (UTRs)).

Given the coding strand sequence encoding the NALP polypeptide disclosed herein (e.g., SEQ ID NO:2 or SEQ ID NO:4), antisense nucleic acids of the invention are designed according to the rules of Watson and Crick base pairing. The antisense nucleic acid molecule is complementary to the entire coding region of NALP1 or NALP5 mRNA, but more preferably is an oligonucleotide which is antisense to only a fragment of the coding or noncoding region of NALP1 or NALP5 mRNA. For example, an antisense oligonucleotide is complementary to the region surrounding the translation start site of NALP1 mRNA, such as the antisense RNA sequence 5′-TTAAGAGGGTGTCTGGGGGATGTT (SEQ ID NO:5), which is complementary to a region −14 to −38 bases upstream (i.e., 5′) of the AUG start codon of SEQ ID NO:1.

An antisense oligonucleotide is, for example, about 5, 10, 15, 20, 25, 30, 35, 40, 45 or 50 nucleotides in length. An antisense nucleic acid of the invention is constructed using chemical synthesis and enzymatic ligation reactions using procedures known in the art. For example, an antisense nucleic acid (e.g., an antisense oligonucleotide) is chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Alternatively, the antisense nucleic acid is produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules of the invention are typically administered to a subject or generated in situ such that they hybridize with or bind to cellular mRNA and/or genomic DNA encoding a NALP, preferably a NALP1 or NALP5 polypeptide to thereby inhibit expression of the polypeptide, e.g., by inhibiting transcription and/or translation. The hybridization is by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule is modified to target selected cells and then administered systemically. For example, for systemic administration, an antisense molecule is modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule is delivered to cells using the vectors described herein.

In a preferred embodiment, NALP gene expression is inhibited using RNA interference (RNAi). This is a technique for post-transcriptional gene silencing (PTGS), in which target gene activity is specifically abolished with cognate long double-stranded RNA (dsRNA) or short interfering RNA (siRNA). RNAi resembles in many aspects PTGS in plants and has been detected in many invertebrates including trypanosome, hydra, planaria, nematode and fruit fly (Drosophila melanogaster). RNAI in mammalian systems is disclosed in International Application No. WO 00/63364 which is incorporated by reference herein in its entirety. Basically, dsRNA of at least about 600 nucleotides or siRNA of about 20 nucleotides (e.g., see Example 1), homologous to the target (NALP1 or NALP5) is introduced into the cell and a sequence specific reduction in gene activity is observed (Example 5). Tuschl's rules are also used in the invention for selecting a siRNA to inhibit the expression of a NALP1 or NALP5 gene or polynucleotide. Tuschl's rules are well known in the art for preparing siRNAs and have been described in detail (see, Elbashir et al., 2003).

B. NALP1 and NALP5 Polypeptides

In certain embodiments, the invention is directed to methods for screening compounds which inhibit NALP polypeptide activity. In other embodiments, the invention is directed to methods for screening compounds which modulate apoptosis in a mammalian cell via inhibiting NALP polypeptide activity. In other embodiments, the invention is directed to NAPL1 and/or NALP5 polypeptides comprising a mutation in the NBS.

Thus, in particular embodiments, the present invention provides isolated and purified NALP1 and NALP5 polypeptides, or fragments thereof. Preferably, a full length polypeptide of the invention is a recombinant polypeptide. Typically, a NALP1 polypeptide or a NALP5 polypeptide is produced by recombinant expression in a prokaryotic host cell or a eukaryotic host cell, preferably a mammalian host cell. A NALP1 or NALP5 polypeptide fragment of the invention is recombinantly expressed or prepared via peptide synthesis methods known in the art (Barany et al., 1987; U.S. Pat. No. 5,258,454). As defined hereinafter, the terms “polypeptide” and “protein” are used interchangeably, both of which refer to about 25 or more amino acids covalently linked via a substituted amide linkage (i.e., a peptide bond).

The amino acid sequence of a human NALP1 polypeptide is represented as SEQ ID NO:2 and the amino acid sequence of a human NALP5 polypeptide is represented as SEQ ID NO:4. A NALP1 polypeptide or NALP5 polypeptide of the invention includes any functional variants of a human NALP1 or NALP5 polypeptide. Functional allelic variants are naturally occurring amino acid sequence variants of a human NALP1 polypeptide or NALP5 polypeptide that maintain the ability to activate a caspase protein and/or modulate apoptosis. Functional allelic variants will typically contain only conservative substitutions of one or more amino acids, or substitution, deletion or insertion of non-critical residues in non-critical regions of the polypeptide.

The invention further provides non-human orthologues of a human NALP1 or NALP5 polypeptide. Orthologues of human NALP1 or NALP5 are polypeptides that are isolated from non-human organisms and possess the same ligand binding (e.g., ATP, a caspase) and signaling capabilities (e.g., pro-apoptosis) as a human NALP1 or NALP5 polypeptide. Orthologues of a human NALP1 or NALP5 polypeptide are identified as comprising an amino acid sequence that is substantially homologous to SEQ ID NO:2 or SEQ ID NO:4.

As used herein, two proteins are substantially homologous when the amino acid sequence of the two proteins (or a region of the proteins) are at least about 60-65%, typically at least about 70-75%, more typically at least about 80-85%, and most typically at least about 90-95% or more homologous to each other.

To determine the percent homology of two amino acid sequences (or of two nucleic acids), the sequences are aligned for optimal comparison purposes (e.g., gaps can be introduced in the sequence of one protein for optimal alignment with the other protein). The amino acid residues at corresponding amino acid positions are then compared. When a position in one sequence is occupied by the same amino acid residue as the corresponding position in the other sequence, then the molecules are homologous at that position (i.e., as used herein amino acid (or nucleic acid) “homology” is equivalent to amino acid (or nucleic acid) “identity”). The percent homology between the two sequences is a function of the number of identical positions shared by the sequences (i.e., % homology=number of identical positions/total number of positions×100).

Modifications and changes can be made in the structure of a NALP polypeptide of the present invention and still obtain a molecule having NALP1 or NALP5 pro-apoptotic activity. For example, certain amino acids can be substituted for other amino acids in a sequence without appreciable loss of pro-apoptotic activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids are considered. The importance of the hydropathic amino acid index in conferring interactive biologic function on a polypeptide is generally understood in the art (e.g. see Kyte & Doolittle, 1982).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those which are within +/−1 are particularly preferred, and those within +/−0.5 are even more particularly preferred.

Substitution of like amino acids can also be made on the basis of hydrophilicity, particularly where the biological functional equivalent polypeptide, or peptide thereby created, is intended for use in immunological embodiments. U.S. Pat. No. 4,554,101, incorporated by reference herein in its entirety, states that the greatest local average hydrophilicity of a polypeptide, as governed by the hydrophilicity of its adjacent amino acids, correlates with its immunogenicity and antigenicity, i.e. with a biological property of the polypeptide.

As set forth above, amino acid substitutions are generally therefore based on the relative similarity of the amino acid side-chain substituents, for example, their hydrophobicity, hydrophilicity, charge, size, and the like. Exemplary substitutions which take of the foregoing various characteristics into consideration are well known to those of skill in the art and include: arginine and lysine; glutamate and aspartate; serine and threonine; glutamine and asparagine; and valine, leucine and isoleucine (see Table 2 below). The present invention thus contemplates functional or biological equivalents of a polypeptide as set forth above.

TABLE 2
EXEMPLARY AMINO ACID SUBSTITUTIONS

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

Biological or functional equivalents of a polypeptide are also prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. As noted above, such changes are desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

It is contemplated in the present invention, that a NALP1 polypeptide or a NALP5 polypeptide is advantageously cleaved into fragments for use in further structural or functional analysis, or in the generation of reagents such as NALP1- or NALP5-related polypeptides and antibodies. This is accomplished by treating purified or unpurified polypeptide with a protease such as glu-C (Boehringer, Indianapolis, Ind.), trypsin, chymotrypsin, V8 protease, pepsin and the like. Treatment with CNBr is another method by which NALP fragments are produced from natural NALP polypeptides. Recombinant techniques are also used to express specific fragments (e.g., a NBS domain) of a NALP1 or a NALP5 polypeptide. For example, in certain embodiments the invention provides recombinantly expressed NALP1 NBS domains (i.e., amino acid residues 328-637 of SEQ ID NO:2) and/or NALP5 NBS domains (i.e., amino acid residues 191-510 of SEQ ID NO:4) or NBS or Mg²⁺ mutants thereof.

In addition, the invention also contemplates that compounds sterically similar to NALP1 or NALP5 may be formulated to mimic the key portions of the peptide structure, called peptidomimetics or peptide mimetics. Mimetics are peptide-containing molecules which mimic elements of polypeptide secondary structure. See, for example, Johnson et al. (1993) and U.S. Pat. No. 5,817,879. The underlying rationale behind the use of peptide mimetics is that the peptide backbone of polypeptides exists chiefly to orient amino acid side chains in such a way as to facilitate molecular interactions, such as those of receptor and ligand.

Successful applications of the peptide mimetic concept have thus far focused on mimetics of β-turns within polypeptides. β-turn structures within a NALP1 or NALP5 polypeptide are predicted by computer-based algorithms. U.S. Pat. No. 5,933,819 describes a neural network based method and system for identifying relative peptide binding motifs from limited experimental data. In particular, an artificial neural network (ANN) is trained with peptides with known sequences and function (i.e., binding strength) identified from a phage display library. The ANN is then challenged with unknown peptides and predicts relative binding motifs. Analysis of the unknown peptides validate the predictive capability of the ANN. Once the component amino acids of the turn are determined, mimetics are constructed to achieve a similar spatial orientation of the essential elements of the amino acid side chains, as discussed in Johnson et al. (1993); U.S. Pat. No. 6,420119 and U.S. Pat. No. 5,817,879.

In certain embodiment, the invention provides recombinantly expressed NALP1 and NALP5 fusion polypeptides. As defined hereinafter, a “fusion polypeptide” (also known as “chimeric” or “hybrid” proteins) is encoded by two or more, often unrelated, fused genes or fragments thereof. Fusion polypeptides and their many uses are well known in the art (e.g., see Thorner et al., 2000(a) and Thorner et al., 2000(b)). Thus, in certain embodiments, the invention is directed to a NALP1 and/or NALP5 fusion polypeptide comprising an N-terminal or a C-terminal epitope tag. An epitope tag typically comprises a relatively short amino acid sequence recognized by a preexisting antibody. Examples of such epitope tags contemplated for use in the present invention include, but are not limited to, myc, HA, FLAG, polyhistidine, AU1, AU5, IRS, B-tag, universal, S-tag, protein C, Glu-Glu, KT3, VSV, T7 and HSV (Thorner et al., 2000(b)).

In other embodiments, a fusion polypeptide of the invention comprises a NALP1 or a NALP5 polypeptide fused to a “reporter protein”, wherein the reporter protein is used for detecting NALP-reporter gene expression in a prokaryotic cell or a eukaryotic cell. For example, a “reporter protein” for detecting gene expression in a prokaryotic cell includes proteins such as a lacZ, alkaline phosphatase and Green Fluorescent Protein (GFP). In certain preferred embodiments, a eukaryotic host cell of the invention is a mammalian cell, more preferably a human cell. Thus, in these embodiments, it is contemplated that a NALP1-reporter fusion or a NALP5-reporter fusion comprises a reporter fusion such as luciferase, aequorin, GFP, enhanced GFP (EGFP), the β-galactosidase/1,2 dioxetane system, the β-glucuronidase/glucuron 1,2-dioxetane system, the placental alkaline phosphatase system, CAT, β-lactamase, and the like.

C. Vectors, Host Cells and Recombinant Polypeptides

In an alternate embodiment, the present invention provides expression vectors comprising polynucleotides that encode NALP1 and/or NALP5 polypeptides. Preferably, the expression vectors of the invention comprise polynucleotides operatively linked to an enhancer-promoter. In certain embodiments, the expression vectors of the invention comprise polynucleotides operatively linked to a prokaryotic promoter. Alternatively, the expression vectors of the present invention comprise polynucleotides operatively linked to an enhancer-promoter that is a eukaryotic promoter, and the expression vectors further comprise a polyadenylation signal that is positioned 3′ of the carboxy-terminal amino acid and within a transcriptional unit of the encoded polypeptide.

Expression of proteins in prokaryotes is most often carried out in E. coli with vectors containing constitutive or inducible promoters directing the expression of either fusion or non-fusion proteins. Fusion vectors add a number of amino acids to a protein encoded therein, to the amino or carboxy terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith and Johnson, 1988), pMAL (New England Biolabs, Beverly; MA) and pRIT5 (Pharmacia, Piscataway, N.J.) which fuse glutathione S-transferase (GST), maltose E binding protein, or protein A, respectively, to the target recombinant protein. For a detailed review of fusion systems, including expression vectors, see Thorner et al., 2000(a); Thorner et al., 2000(b) and Thorner et al., 2000(c).

Examples of suitable inducible, non-fusion E. coli expression vectors include pTrc (Amann et al., 1988) and pET lid (Studier et al., 1990). Target gene expression from the pTrc vector relies on host RNA polymerase transcription from a hybrid trp-lac fusion promoter. Target gene expression from the pET lid vector relies on transcription from a T7 gnl β-lac fusion promoter mediated by a coexpressed viral RNA polymerase T7 gnl. This viral polymerase is supplied by host strains BL21 (DE3) or HMS 174(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention can be carried out by standard DNA mutagenesis or synthesis techniques.

In another embodiment, a polynucleotide of the invention is expressed in mammalian cells using a mammalian expression vector. Examples of mammalian expression vectors include pCDM8 (Seed, 1987) and pMT2PC (Kaufman et al., 1987). When used in mammalian cells, the expression vector's control functions are often provided by viral regulatory elements.

For example, commonly used promoters are derived from polyoma virus, Adenovirus 2, cytomegalovirus (CMV) and Simian Virus 40 (SV40). For other suitable expression systems for both prokaryotic and eukaryotic cells see chapters 16 and 17 of Sambrook et al., “Molecular Cloning: A Laboratory Manual” 2nd, ed, Cold Spring Harbor Laboratory, Cold Spring Harbor Laboratory Press, Cold Spring Harbor, N.Y., 1989, incorporated herein by reference.

A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. A preferred transcription-terminating region used in an adenovirus vector construct of the present invention comprises a polyadenylation signal of SV40 or the protamine gene. Listed in Table 3 and Table 4 are non-limiting examples of tissue specific and inducible promoters contemplated for use.

TABLE 3
TISSUE SPECIFIC PROMOTERS

| PROMOTER | Target |
|---|---|
| Tyrosinase | Melanocytes |
| Tyrosinase Related Protein, | Melanocytes |
| TRP-1 | |
| Prostate Specific Antigen, | Prostate Cancer |
| PSA | |
| Albumin | Liver |
| Apolipoprotein | Liver |
| Plasminogen Activator | Liver |
| Inhibitor Type-1, PAI-1 | |
| Fatty Acid Binding | Colon Epithelial Cells |
| Insulin | Pancreatic Cells |
| Muscle Creatine Kinase, | Muscle Cell |
| MCK | |
| Myelin Basic Protein, MBP | Oligodendrocytes and |
| | Glial Cells |
| Glial Fibrillary Acidic | Glial Cells |
| Protein, GFAP | |
| Neural Specific Enolase | Nerve Cells |
| Immunoglobulin Heavy | B-cells |
| Chain | |
| Immunoglobulin Light Chain | B-cells, |
| | Activated T-cells |
| T-Cell Receptor | Lymphocytes |
| HLA DQα and DQβ | Lymphocytes |
| β-Interferon | Leukocytes; |
| | Lymphocytes Fibroblasts |
| Interlukin-2 | Activated T-cells |
| Platelet Derived Growth | Erythrocytes |
| Factor | |
| E2F-1 | Proliferating Cells |
| Cyclin A | Proliferating Cells |
| α-, β-Actin | Muscle Cells |
| Haemoglobin | Erythroid Cells |
| Elastase I | Pancreatic Cells |
| Neural Cell Adhesion | Neural Cells |
| Molecule, NCAM | |

TABLE 4
Inducible Promoters

| PROMOTER ELEMENT | INDUCER |
|---|---|
| Early Growth Response-1 | Radiation |
| Gene, egr-1 | |
| Tissue Plasmingen | Radiation |
| Activator, t-PA | |
| fos and jun | Radiation |
| Multiple Drug Resistance | Chemotherapy |
| Gene 1, mdr-1 | |
| Heat Shock Proteins; | Heat |
| hsp16, hs60, hps68, | |
| hsp70, | |
| human Plasminogen | Tumor Necrosis Factor, |
| Activator Inhibitor type-1, | TNF |
| hPAI-1 | |
| Cytochrome P-450 | Toxins |
| CYP1A1 | |
| Metal-Responsive | Heavy Metals |
| Element, MRE | |
| Mouse Mammary Tumor | Glucocorticoids |
| Virus | |
| Collagenase | Phorbol Ester |
| Stromolysin | Phorbol Ester |
| SV40 | Phorbol Ester |
| Proliferin | Phorbol Ester |
| α-2-Macroglobulin | IL-6 |
| Murine MX Gene | Interferon, Newcastle |
| | Disease Virus |
| Vimectin | Serum |
| Thyroid Stimulating | Thyroid Hormone |
| Hormone α Gene | |
| HSP70 | Ela, SV40 Large T |
| | Antigen |
| Tumor Necrosis Factor | FMA |
| Interferon | Viral Infection, dsRNA |
| Somatostatin | Cyclic AMP |
| Fibronectin | Cyclic AMP |

The invention further provides a recombinant expression vector comprising a DNA molecule encoding a NALP1 or NALP5 polypeptide cloned into the expression vector in an antisense orientation. That is, the DNA molecule is operatively linked to a regulatory sequence in a manner which allows for expression (by transcription of the DNA molecule) of an RNA molecule which is antisense to NALP1 or NALP5 mRNA. Regulatory sequences operatively linked to a nucleic acid cloned in the antisense orientation are chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

Another aspect of the invention pertains to host cells into which a recombinant expression vector of the invention has been introduced. The terms “host cell”, “genetically engineered” host cell and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, the polypeptide can be expressed in bacterial cells such as E coli, insect cells, yeast or mammalian cells (such as Chinese hamster ovary cells (CHO), COS cells, NIH-3T3 cells, HeLa cells, NOS cells or PER-C6 cells). Other suitable host cells are known to those skilled in the art.

Vector DNA is introduced into prokaryotic or eukaryotic cells via conventional transformation, infection or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (“Molecular Cloning: A Laboratory Manual” 2nd ed, Cold Spring Harbor Laboratory, Cold Spring Harbor Laboratory Press, Cold Spring Harbor, N.Y., 1989), and other laboratory manuals.

A host cell of the invention, such as a prokaryotic or eukaryotic host cell in culture, is used to produce (i.e., express) NALP1 or NALP5 polypeptides. Accordingly, the invention further provides methods for producing polypeptides using the host cells of the invention. In one embodiment, the method comprises culturing the host cell of invention (into which a recombinant expression vector encoding a polypeptide has been introduced) in a suitable medium until the polypeptide is produced. In another embodiment, the method further comprises isolating the polypeptide from the medium or the host cell. In other embodiments, the host cell expressing polypeptide is assayed for apoptosis or screened for compounds which inhibit apoptosis.

An expression vector of the present invention is useful both as a means for preparing quantities of the polypeptide-encoding DNA itself, and as a means for preparing the encoded polypeptide and peptides. It is contemplated that where polypeptides of the invention are made by recombinant means, one can employ either prokaryotic or eukaryotic expression vectors as shuttle systems. However, prokaryotic systems are usually incapable of correctly processing precursor polypeptides and, in particular, such systems are incapable of correctly processing membrane associated eukaryotic polypeptides, and since eukaryotic polypeptides are anticipated using the teaching of the disclosed invention, one likely expresses such sequences in eukaryotic hosts. However, even where the DNA segment encodes a eukaryotic polypeptide, it is contemplated that prokaryotic expression can have some additional applicability. Therefore, the invention can be used in combination with vectors which can shuttle between the eukaryotic and prokaryotic cells. Such a system is described herein which allows the use of bacterial host cells as well as eukaryotic host cells.

Where expression of recombinant polypeptides is desired and a eukaryotic host is contemplated, it is most desirable to employ a vector such as a plasmid, that incorporates a eukaryotic origin of replication. Additionally, for the purposes of expression in eukaryotic systems, one desires to position the encoding sequence adjacent to and under the control of an effective eukaryotic promoter such as promoters used in combination with Chinese hamster ovary cells. To bring a coding sequence under control of a promoter, whether it is eukaryotic or prokaryotic, what is generally needed is to position the 5′ end of the translation initiation side of the proper translational reading frame of the polypeptide between about 1 and about 50 nucleotides 3′ of, or downstream, of the promoter chosen. Furthermore, where eukaryotic expression is anticipated, one would typically desire to incorporate into the transcriptional unit, which includes the polypeptide, an appropriate polyadenylation site.

The pCMV plasmids are a series of mammalian expression vectors of particular utility in the present invention. The vectors are designed for use in essentially all cultured cells and work extremely well in SV40-transformed simian COS cell lines. The pCMV1, 2, 3, and 5 vectors differ from each other in certain unique restriction sites in the polylinker region of each plasmid. The pCMV4 vector differs from these four plasmids in containing a translation enhancer in the sequence prior to the polylinker. While they are not directly derived from the pCMV1-5 series of vectors, the functionally similar pCMV6b and pCMV6c vectors are available from the Chiron Corp. (Emeryville, Calif.) and are identical except for the orientation of the polylinker region which is reversed in one relative to the other.

The universal components of the pCMV plasmids are as follows. The vector backbone is pTZ18R (Pharmacia), and contains a bacteriophage f1 origin of replication for production of single stranded DNA and an ampicillin-resistant gene. The CMV region consists of nucleotides −760 to +3 of the powerful promoter-regulatory region of the human cytomegalovirus (Towne stain) major immediate early gene (Thomsen et al., 1984; Boshart et al., 1985). The human growth hormone fragment (hGH) contains transcription termination and poly-adenylation signals representing sequences 1533 to 2157 of this gene (Seeburg, 1982). There is an Alu middle repetitive DNA sequence in this fragment. Finally, the SV40 origin of replication and early region promoter-enhancer derived from the pcD-X plasmid (HindIII to Pstl fragment) described in (Okayama et al., 1983). The promoter in this fragment is oriented such that transcription proceeds away from the CMV/hGH expression cassette.

The pCMV plasmids are distinguishable from each other by differences in the polylinker region and by the presence or absence of the translation enhancer. The starting pCMV1 plasmid has been progressively modified to render an increasing number of unique restriction sites in the polylinker region. To create pCMV2, one of two EcOR1 sites in pCMV1 were destroyed. To create pCMV3, pCMV1 was modified by deleting a short segment from the SV40 region (Stul to EcOR1), and in so doing made unique the Pstl, Sal, and BamHl sites in the polylinker. To create pCMV4, a synthetic fragment of DNA corresponding to the 5′-untranslated region of an mRNA transcribed from the CMV promoter was added. The sequence acts as a translational enhancer by decreasing the requirements for initiation factors in polypeptide synthesis. To create pCMV5, a segment of DNA (Hpal to EcOR1) was deleted from the SV40 origin region of pCMV1 to render unique all sites in the starting polylinker.

The pCMV vectors have been successfully expressed in simian COS cells, mouse L cells, CHO cells, and HeLa cells. In several side by side comparisons they have yielded 5- to 10-fold higher expression levels in COS cells than SV40-based vectors. The pCMV vectors have been used to express the LDL receptor, nuclear factor 1, GS a polypeptide, polypeptide phosphatase, synaptophysin, synapsin, insulin receptor, influenza hemagglutinin, androgen receptor, sterol 26-hydroxylase, steroid 17- and 21-hydroxylase, cytochrome P-450 oxidoreductase, β-adrenergic receptor, folate receptor, cholesterol side chain cleavage enzyme, and a host of other cDNAs. It should be noted that the SV40 promoter in these plasmids can be used to express other genes such as dominant selectable markers. Finally, there is an ATG sequence in the polylinker between the HindIII and Pstl sites in pCMU that can cause spurious translation initiation. This codon should be avoided if possible in expression plasmids. A paper describing the construction and use of the parenteral pCMV1 and pCMV4 vectors has been published (Anderson et al., 1989b).

In yet another embodiment, the present invention provides recombinant host cells transformed, infected or transfected with polynucleotides that encode polypeptides. Means of transforming or transfecting cells with exogenous polynucleotide such as DNA molecules are well known in the art and include techniques such as calcium-phosphate- or DEAE-dextran-mediated transfection, protoplast fusion, electroporation, liposome mediated transfection, direct microinjection and adenovirus infection (Sambrook, Fritsch and Maniatis, 1989).

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

D. Uses of the Invention

The polynucleotides, polypeptides and host cells of the invention are used in one or more of the following methods: a) drug screening assays; b) diagnostic assays, particularly in apoptotic disease identification; c) methods of treatment; and d) monitoring of effects during clinical trials. A polypeptide of the invention (e.g., NALP1 or NALP5) is used as a drug target for developing agents (e.g., small molecules, peptide mimetics) to inhibit NALP1 and/or NALP5 activity. Similarly an antisense RNA molecule or a siRNA molecule is used to modulate NALP1 or NALP5 expression, thereby reducing NALP polypeptide levels (e.g., see Example 5).

Thus, the invention provides methods for identifying compounds or agents that inhibit NALP1 or NALP5 polypeptide activity. These methods are also referred to herein as drug screening assays. In certain embodiments, the invention is directed to a method for screening compounds which inhibit NALP polypeptide activity comprising the steps of (a) providing a host cell comprising a polynucleotide expressing a NALP polypeptide; (b) contacting the cell with a test compound; and (c) assaying caspase activity, wherein a decrease in caspase activity indicates the test compound inhibits NALP activity.

Candidate/test compounds include, for example, (1) small molecules, organic and inorganic (e.g., molecules obtained from combinatorial and natural product libraries); (2) peptides such as soluble peptides, including Ig-tailed fusion peptides and members of random peptide libraries and combinatorial chemistry-derived molecular libraries made of D-configuration and/or L-configuration amino acids; (3) phosphopeptides (e.g., members of random and partially degenerate directed phosphopeptide libraries; Songyang et al., 1993); (4) peptide mimetics; (5) antibodies (e.g., polyclonal, monoclonal, humanized, anti-idiotypic, chimeric, and single chain antibodies, as well as Fab, F(ab′)₂, Fab expression library fragments, and epitope-binding fragments of antibodies) and (6) nucleic acid molecules such as antisense RNA, dsRNA and siRNA.

In certain embodiments, the invention is directed to methods for screening compounds which inhibit the activity of a NALP polypeptide and thereby reduce or inhibit an apoptotic pathway. Apoptosis is measured by direct visualization, flow cytometry (propidium iodide labeling), measuring the expression of Fas, detecting DNA fragmentation, (e.g., using an In Situ ApopTag™ kit (Talron Scientific & Medical Products Ltd., Israel)) and by detecting post translational ε(γ-glutamyl)lysine isodopeptide bond formation. U.S. Pat. No. 5,750,360, incorporated herein by referenced in its entirety, describes methods for detecting concentrations of isodopeptide as low as 25 pmol.

In other embodiments, caspase activity is measured as a screen for compounds which inhibit a NALP polypeptide of the invention. Caspases are responsible for the degradation of cellular proteins that leads to the morphological changes seen in cells undergoing apoptosis. Caspases are cysteine proteases having specificity for aspartate the substrate cleavage site. In a preferred embodiment, caspase activity is measured by detecting the cleavage of a fluorescently labeled caspase substrate, wherein fluorescence emission of the fluorophore (i.e., the substrate) increases upon cleavage by the caspase. In a preferred embodiment, caspase-3 activity is assayed in a host cell expressing a NALP1 and/or a NALP5 polypeptide. In a preferred embodiment, a caspase-3 substrate cleavage site, represented as a single letter amino acid code, is DEVD (SEQ ID NO:6). In other embodiments, a caspase-3 substrate cleavage site, represented as a single letter amino acid code, is SHVD (SEQ ID NO:7), DBLD (SEQ ID NO:8), DGPD (SEQ ID NO:9), DEPD (SEQ ID NO:10), DGTD (SEQ ID NO:11), DLND (SEQ ID NO:12), DEED (SEQ ID NO:13), DSLD (SEQ ID NO:14) or DVPD (SEQ ID NO:15), wherein caspase-3 has a lower affinity for these substrate sties relative to DEVD. In still other embodiments, the activity of caspase-1, caspase-2, caspase-4, caspase-5, caspase-6, caspase-7, caspase-8 or caspase-9 is assayed. Listed in Table 5 are the optimal substrate sequences for the caspase proteins set forth above. U.S. Pat. No. 6,335,429 and U.S. Pat. No. 6,248,904 (each incorporated herein by reference in its entirety) describe fluorophores and their applications for whole-cell fluorescence screening assays for caspases.

TABLE 5
OPTIMAL CASPASE SUBSTRATE SEQUENCES

| Enzyme* | Optimal Sequence** |
|---|---|
| caspase-1 (ICE) | WEHD (SEQ ID NO: 16) |
| caspase-2 (ICH-1, mNEDD2) | DEHD (SEQ ID NO: 17) |
| caspase-3 (apopain, CPP-32, YAMA) | DEVD (SEQ ID NO: 6) |
| caspase-4 (ICEᵣₑ II, TX, ICH-2) | WEHD (SEQ ID NO: 16) or |
| | LEHD (SEQ ID NO: 18) |
| caspase-5 (ICEᵣₑ III, TY) | WEHD (SEQ ID NO: 16) or |
| | LEHD (SEQ ID NO: 18) |
| caspase-6 (Mch2) | VEHD (SEQ ID NO: 19) |
| caspase-7 (Mch-3, ICE-LAP3, CMH-1) | DEVD (SEQ ID NO: 6) |
| caspase-8 (MACH, FLICE, Mch5) | LETD (SEQ ID NO: 20) |
| caspase-9 (ICE-LAP6, Mch6) | LEHD (SEQ ID NO: 21) |
| granzyme B | IEPD (SEQ ID NO: 22) |

*Enzymes are identified by both new and old (in parentheses) nomenclature.
**Standard one-letter abbreviations for amino acids are used to indicate the optimal amino acid sequences.

In certain embodiments, the invention is directed to methods for detecting neuron damage in a mammalian subject. In certain of these embodiments, the methods include obtaining a biological sample from the subject. As defined hereinafter, the term “biological sample” is intended to include tissues, cells and biological fluids isolated from a subject. In preferred embodiments, the biological sample is selected from the group consisting of blood plasma, serum, erythrocytes, leukocytes, platelets, lymphocytes, macrophages, fibroblast cells, mast cells, fat cells, epithelial cells, nerve cells, glial cells, Schwann cells, progenitor stem cells, a cerebrospinal fluid (CSF), saliva, a skin biopsy, a brain biopsy and a buccal biopsy.

In certain embodiments, it is contemplated that the small molecules, nucleic acids, polypeptides, peptide fragments, and the like (referred to herein as “active compounds”) of the invention are incorporated into pharmaceutical compositions suitable for administration to a subject, e.g., a human. Such compositions typically comprise the nucleic acid molecule, protein, modulator, or inhibitor molecule and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer. Systemic administration can also be by transmucosal or transdermal means. For transmucosal or transdermal administration, penetrants appropriate for the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams, as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated by reference herein in its entirety.

All patents and publications cited herein are incorporated by reference.

### E. EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### Materials and Methods

Preparation and gene expression analysis of injured neurons. Cerebellar granule neurons (CGN) were isolated and cultured from 7-day old rat pups (Miller and Johnson, 1996) and cortical neurons from El 8 rat embryo forebrain (Bossenmeyer-Pourie et al., 1999). CGNs cultured in standard growth medium containing 0.5% serum and 25 mM K⁺ were challenged by transfer into medium without serum and with 5 mM K⁺ for 24 hours. Total RNA was purified from cells using the RNeasy kit (Qiagen, Inc., Valencia, Calif.) per manufacturer's instructions. In the transient focal ischemia experiments, adult male spontaneously hypertensive rats (SHR, Taconic Farms) weighing 280-300 g were anesthetized with 3% isoflurane in 95% O₂/5% CO₂ through a nose cone. Temperature was maintained at 37° C. throughout the surgery using a heating lamp. Transient middle cerebral artery occlusion (MCAO) was induced for 1 hour using an intraluminal suture method (Longa et al., 1989). Briefly, an 18 mm length of 4-0 monofilament nylon suture coated with poly-L-lysine (Belayev et al., 1996) and a flame-rounded tip was inserted into the external carotid artery and advanced through the internal carotid to occlude the origin of the middle cerebral artery. Sixty minutes later the rats were re-anesthesized and the suture was withdrawn. Sham-operated rats were subject to the same surgery, but without advancement of the suture into the middle cerebral artery. At indicated time points post-MCAO, animals were sacrificed by decapitation and right (ischemic, ipsilateral) cortex and left (contralateral) cortex immediately dissected and frozen. Total RNA was extracted using RNAzol B reagent (TEL-TEST, Inc., Friendswood, Tex.). RNA samples were transcribed to first-strand cDNA using SuperScript™ II (Invitrogen Corp., Carlsbad, Calif.) with oligo-dT primer. Five percent of cDNA generated from 1 μg RNA was used per PCR with HotStarTaq (Qiagen) reagents. Positive control reactions used 100 ng rat genomic DNA. PCR cycles were 95° 15 minutes, (94° C. for 30 seconds, 55° C. for 30 seconds, 70° C. for 30 seconds)×35 cycles, 70° C. for 10 minutes. Twenty percent of each reaction mixture was examined by ultraviolet illumination following electrophoresis in 2% agarose tris acetate, ethidium bromide gels at 150 V for 10 minutes. Rat cDNA sequences for ASC, CARD8 and CARD9 were used to design primers to amplify a 73 or 74 base pair product contained within a single genomic exon. The specific segments are: ASC (NM_{—}172322; 125-197) CARD8 (A1044039; (31-104) and CARD9, (NM_{—}022303; 283-356). Rat sequences for NALP1 and NALP5 were not present in GenBank. Therefore, human cDNA sequences were aligned to mouse genomic data using BLAST (Altschul et al., 1990), and the largest mouse exon within protein coding sequences was used to design PCR primers. Using rat genomic DNA as a template, the mouse primers (sense-5′-GGACCAGMTCCTGAGCTGTGT (SEQ ID NO:23) and anti-sense-5′-GAAGCCTCAGGMGGATGGAT (SEQ ID NO:24)) amplified a product of the expected length. These products were cloned into TOPO®-TA vector (Invitrogen) and the nucleotide sequences determined. These DNAs were subsequently utilized to design PCR primers for investigations of expression of NALP1 and NALP5 in rat samples.

Cloning and expression plasmids. The protein-coding region of human NALP1 cDNA was isolated and cloned into mammalian expression vectors as follows. NALP1 was amplified by PCR (Advantage™ GC-rich kit; Clontech (BD Biosciences), Palo Alto, Calif.) in two segments. The amino-terminal coding sequences were generated from human leukocyte Quick-Clone™ cDNA (Clontech) using a primer approximately 70 base pairs 5′ of the translation start site. The carboxy-terminal coding sequences were amplified from human placenta Quick-Clone™ cDNA (Clontech) using a primer approximately 450 base pairs beyond the translational stop site. In both reactions, a primer spanned a natural HindIII site 2295 base pairs into the coding region. The two PCR products were then used as templates to amplify similar DNA molecules adding either a Kpnl site at the NALP15′ end or a Xhol site at the 3′ end. Each was ligated into the TOPO-TA vector, then sequentially moved into pcDNA3.1-myc-His (Invitrogen). The vector was cleaved with Kpnl plus HindIII to accept Kpnl-HindIII-cleaved PCR product and this intermediate plasmid DNA was isolated. This construct was subsequently cleaved with HindIII plus Xhol and the second PCR product was ligated into it to generate full-length NALP1 in frame with the 3′ epitopes. In a similar manner, a construct was generated containing NALP1 in frame with EGFP. The first two PCR products described above were used as templates to generate amino- and carboxy-half molecules containing Xhol (5′) or KpnI (3′) ends, respectively. Each was ligated into the TOPO-TA vector and sequentially moved into pEGFP—N3 (Clontech) to generate full-length NALP1 in frame with EGFP. Full length NALP5 was amplified by PCR from a human cDNA library pool from multiple tissues (Quickclone™ cDNA, Clontech). Primers were designed to isolate the predicted open reading frame according to the cDNA sequence predicted from human genomic DNA. Forward primer was 5′-EcOR1-ATCAAGATGGAAGGAGACAAATCG (SEQ ID NO:25) and the reverse primer was 5′ Apal-GTTTTTCCACCAGTACCGGTC (SEQ ID NO:26). A PCR product of the predicted size (3.1 kb) was isolated and the nucleotide sequence determined. A single clone identical to the predicted NALP5 sequence was subcloned into pcDNA3.1-mycHisA (Invitrogen) or pEGFP—N2 (Clontech), both vectors digested with EcOR1+Apal. This strategy resulted in fusion of epitopes to the carboxy terminus of NALP5. Generation of mutations in the predicted nucleotide binding site of NALP1 was performed by olignucleotide-directed mutagenesis according to the manufacturer's protocol (QuickChange XL Mutagenesis Kit; Stratagene). The oligonucleotides 5′ GGCTGCTGGAATTGAGGCGTCAACACTGGCC (SEQ ID NO:27) and its reverse complement were used to generate the G339E and K340A amino acid changes. The three substituted nucleotides are underlined.

Functional assays. HeLa cells were transfected in 24-well tissue culture plates using the Fugene™ reagent (Roche Diagnostics Corp., Indianapolis, Ind.) per manufacturer's instructions. Cells transfected with plasmids expressing proteins tagged with the mycHis6 epitope were stained with the fluorescent DNA dye Hoechst-33342, as described previously (Kajkowski et al., 2001), plus the caspase activity probe sulforhodamine-DEVD-FMK (CaspaTag™, Intergen Co.), then fixed with 1% paraformaldehyde, 0.1% Triton X-100. Samples were then blocked in incubation buffer (PBS, 3% BSA, 0.1% Triton X-100) for 1 hour, followed by incubation with FITC-conjugated anti-myc or anti-His antibody (1:250 dilution, Invitrogen) in incubation buffer for 1 hour. Samples were mounted in antifade (Molecular Probes, Eugene, Oreg.) and analyzed by fluorescence microscopy. Cerebellar granule neurons (CGN) were isolated from 7-day old pups (Miller and Johnson, 1996) and plated on glass coverslips coated with poly-lysine in 6-well plates. Transfection of CGN was performed day 4 post-isolation, using a modified calcium phosphate method (Xia et al., 1996). Briefly, 6 μg DNA was mixed with CaCl₂ and 2× HBSS for 30 minutes before applied to CGNs. Cells were allowed to incubate with the DNA/CaPO₄ precipitate for 90 minutes at room temperature before replacing with growth media and returned to a 37° C. incubator. CGN samples were fixed and stained, as described above, 48 hours after transfection.

siRNA in HeLa cells. Four siRNA duplexes for human NALP1 were designed according to Tuschl's rules (Elbashir et al., 2003) and synthesized (Dharmacon). The four NALP1 siRNAs were initially characterized by cotransfection of siRNA and pNALP1-EGFP into HeLa cells using Lipofectamine Plus (Invitrogen). Cell lysates were generated 24 hours after transfection and subjected to Western blot analysis. Anti-GFP antibody was used to monitor NALP1-EGFP expression. Two of the four duplexes reduced NALP1-EGFP expression substantially. The siRNA which reduced NALP1-EGFP expression the most was chosen for subsequent experiments. The working NALP1 siRNA sequences are sense-5′-dAdAGAGAAGCUGGCCUGAUUAU (SEQ ID NO:28) and the reverse complement sequence 5′-dAdAAUAAUCAGGCCAGCUUCUC (SEQ ID NO:29). Hela cells were transfected with NALP1 siRNA reagent using Lipofectamine Plus. RNA was isolated 48 hours after transfection from cells using the RNeasy system (Qiagen). One μg of each RNA sample was transcribed to cDNA using the Superscript II kit (Invitrogen). PCR amplifications were performed as described previously, with human NALP1 oligonucleotides sense-5′-GATGAGATGAGGCAGGMCTGA (SEQ ID NO:30) and antisense-5′-CAGGAGAAGGCACGCACAAGAG (SEQ ID NO:31). Parallel samples were challenged with 300 nM etoposide for an additional 24 hours and analyzed for caspase-3 activation. Caspase-3 activation was analyzed by Western blot using anti-active caspase 3 (#96615; Cell Signaling).

Production of purified NALP-Flashplate binding assay. NALP1 protein for in vitro binding assays was purified from transiently transfected cells. COS-7 cells were plated in DMEM, 10% fetal calf serum with amino acid supplements and antibiotics and allowed to grow to approximately 50% confluency. Large-scale preparations were performed in Cell Factories (NUNC) with approximately 600 μg plasmid DNA. Polyfect transfection reagent (Qiagen) was used as described by the manufacturer. After 24 hours, cells were collected by trypsinization, washed with fresh medium and then with PBS. Cells were lysed by resuspension in 10× cell volume of lysis buffer (140 mM NaCl, 0.1% Triton-X100, 0.1 mM DTT, 20 mM HEPES at pH 7.4 and protease inhibitor cocktail (Roche Diagnostics BmbH)). Lysates were centrifuged at approximately 7400×g for 30 minutes and supernatants were supplemented with glycerol to a final concentration of 10% and stored at −70° C. until purification. Protein concentrations were measured and samples normalized. Extracts were mixed with 1 ml Ni-NTA agarose equilibrated with the same buffer for 1 hour at 4° C. in the tube. Disposable columns were filled with the resin and flow-through collected. Columns were washed with 20 ml of the same buffer plus 20 mM imidazole to remove weakly bound contaminant proteins. His-tagged proteins were eluted 2× with 1 ml of the same buffer plus 300 mM imidazole. Samples were dialyzed overnight against 1 L buffer (20 mM Tris-HCl pH 7.5, 150 mM NaCl, 10% glycerol). Ni Flashplate Plus (PerkinElmer LifeSciences, Inc) were pre-washed with Buffer A: PBS, 0.01% Triton-X100, 10% glycerol, 0.5 mM DTT. Proteins (100 μl) were then added and incubated at room temperature for 1 hour. Solution was discarded and plates were washed once with Buffer B: PBS, 0.01% Triton-X100, 0.5 mM DTT, 10 mM MgCl₂. Eight nM dATPα³⁵S (1 μCi/well, 1250 Ci/mmol; NEN PerkinElmer) with or without 100 mM non-labeled dATP was prepared in Buffer B and incubated with immobilized protein for 1 hour. Reactions were stopped by aspirating solutions and washed once with Buffer B. Plates were counted in a Top Count scintillation counter (Packard).

Cell extract preparation and fluorimetric assay of caspase activity. Preparation of cell extracts, dATP incubation and caspase-3 activities assays were performed as described by Liu et al. (1996). Briefly, HeLa cells were grown in DMEM, 10% FBS. Cells were plated in T175 flasks to 60% confluence. Control vector pEGFP or wild-type or NBS mutant NALP1-EGFP expression plasmids were transfected into HeLa cells using Lipofectamine Plus. Cells were collected in homogenization buffer (20 mM HEPES pH 7.5, 10 mM KCl, 1 mM sodium EDTA, 1.5 MgCl₂, 1 mM sodium EGTA, 0.1% CHAPS, 1 mM DTT, 0.1 mM PMSF and protease inhibitor cocktail tablets (Roche)), 24 hours after transfection. Cells were chilled on ice for 15 minutes and homogenized by douncing 30 times in a glass douncer. Samples were centrifuged at 1000×g for 10 minutes at 4° C. The supernatant was further centrifuged at 10⁵x g for 30 minutes in a Beckman TLA-45 rotor. The resulting supernatant (S-100 fraction) was collected and used for in vitro caspase activity assays. S100 (˜10 μg/μl) extracts were incubated with or without 2 mM dATP, 2 mM MgCl₂ for 1 hour at 37° C. Fluorometric assays of proteolytic activity were conducted using synthetic fluorogenic substrate (BD PharMingen). Extracts (30 μl) were assayed in 200 μl protease assay buffer (20 mM HEPES pH 7.5, 10% glycerol, 2 mM DTT) with 10 μl substrate, Ac-DEVD-AMC (1 mg/ml), with or without 10 μl inhibitor, Ac-DEVD-CHO (1 mg/ml). After 2 hours incubation, liberation of AMC (7-amino-4-methylcoumarin) from the substrate was monitored using excitation/emission wavelength pairs (λₑₓ/λₑₘ) of 380/460 nm using a SpectraFluor Plus instrument (Tecan). Triplicate samples were evaluated with or without specific inhibitors. Values with inhibitor were subtracted from the values without inhibitor to obtain caspase-specific activities. All calculations were normalized relative to the value (equal to 1) for the vector control reaction. Normalized data from multiple experiments were expressed as means±standard deviation. For each experiment, the expression of EGFP-tagged NALP1 proteins was analyzed by anti-GFP immunoblot and quantified by densitometry.

### Example 2

### Nalp and Card Gene Expression in Injured Neurons

The recently described inflammasome (Martinon et al., 2002) is minimally comprised of NALP1, ASC, and caspase subtypes −1 and −5. NALP1 and the inflammasome appear to be most prominently expressed in immune cells, but expression of the ASC gene in brain samples (Masumoto et al., 1999) and in isolated neurons (data not shown) has also been observed. This observation and the report that NALP1 may also regulate CARD-containing apoptotic caspases such as subtypes −2 and −9 (Chu et al., 2001; Hlaing et al., 2001) prompted an investigation of NALP1 gene expression in cultured primary neurons. In addition to NALP1 and ASC, suggestions of brain expression had been observed for another NALP family member, NALP5, and the apoptosis regulators CARD8 (Razmara et al., 2002) and CARD9 (Pathan et al., 2001). The modular configurations of the protein products of these genes are shown in FIG. 1. It was necessary to obtain rat DNA sequence information for the design and synthesis of PCR primers to investigate gene expression in cultured rat primary neurons.

Rat cDNA sequences for ASC, CARD8 and CARD9 are described in Genbank, but rat information for NALP1 and NALP5 could not be identified by alignments with the known human sequences. Therefore, a strategy was developed to identify mouse DNA sequence information and utilize those results to isolate rat DNA. Human cDNA sequences were aligned with mouse genomic DNA to identify exons of mouse NALP1 and NALP5. PCR primers were then designed to amplify genomic DNA (within a single exon). With the substantial sequence similarity between mouse and rat, it was then possible to obtain PCR amplicons from rat genomic DNA. The DNA sequences of the rat amplicons were determined and subsequently utilized to design PCR primers for rat gene expression analysis. Oligonucleotide primers and intervening rat DNA sequences are described in Example 1, Material and Methods.

A primary interest was to identify potential apoptosis regulatory proteins expressed in neuronal cells, as opposed to immune cells of the brain such as microglia. Therefore, first investigations of NALP and CARD expression patterns were conducted in cultured rat neurons rather than tissue samples containing multiple cell types. Cerebellar granule neurons (CGN) in culture were subjected to injury by transfer to medium lacking serum and containing reduced potassium (K⁺). This treatment of serum/K⁺ withdrawal induces apoptosis in approximately 60% of cells in 24 hours (Chiang et al., 2001). Cortical neurons were also investigated. Freshly cultured cortical neuron samples have a high proportion of cells undergoing apoptosis, as measured by nuclear morphology or caspase-3 induction (data not shown), so these neurons could not be evaluated in a non-apoptotic state. First-strand cDNA was generated from total RNA isolated from the cultured neurons, and used as template for PCRs with the gene-specific primers. The ASC gene was expressed in both cortical and CGN samples, with no substantial change observed in untreated and injured CGNs (FIG. 2). Both NALP1 and NALP5 were weakly expressed in untreated neurons, and showed substantial elevation of expression in the injured CGNs, suggesting a potential function in cell death signaling, or possibly in a protective response. The expression of CARD8 and CARD9 could not be detected (FIG. 2). With the absence of expression in cultured neurons, these genes were dropped from further consideration as intrinsic neuronal apoptosis modulators.

### Example 3

NALP1 AND NALP5 Expression in Transient Focal Ischemia

A portion of the neurodegeneration and brain dysfunction following ischemic stroke results from time-delayed neuronal apoptosis (Li et al., 1995; Namura et al., 1998). To extend the finding that NALP1 and NALP5 gene expression was induced in injured neurons in culture, similar reverse transcription-PCR investigations were conducted using cortical samples isolated from adult rats subjected to transient middle cerebral artery occlusion (MCAO) followed by a time course of reperfusion. In addition to cortical tissue from a rat receiving no occlusion (sham-operated), contralateral hemispheres of animals at each reperfusion time point served as non-injury controls. NALP1, NALP5 and ASC expression was evaluated using the same PCR primers as described previously. GAPDH served as a constitutively expressed control, and HSP70 as an inducible control (Kinouchi et al., 1993). The ASC gene was expressed in both ipsilateral and contralateral samples, with no inducibility apparent in this qualitative assay (FIG. 3). NALP1 and NALP5 mRNAs were not detected in tissue from sham-operated animal, nor in contralateral hemispheres of MCAO rats. This absence contrasts with the weak expression observed in the cultured neurons, possibly resulting from developmental expression or from a cellular stress response to explantation. NALP1 and NALP5 showed substantially elevated expression in MCAO samples (FIG. 3). Specifically, NALP1 expression was elevated at all time points following ischemia and reperfusion, and NALP5 was clearly elevated at 1 hour reperfusion and was detectable at 8 hours. NALP5 expression was not observed in 12 and 24 hour reperfusion samples (FIG. 3). Elevated expression of NALP1 and NALP5 in ischemic brain tissue suggests a potential function in neuronal apoptosis during restoration of nutrient and oxygen flow. The appearance of NALP5 expression at early but not later time points may indicate a function only in the initiating phase of cellular response to injury.

### Example 4

### Induction of Neuronal Apoptosis by Recombinant Expression of NALP1 and NALP5

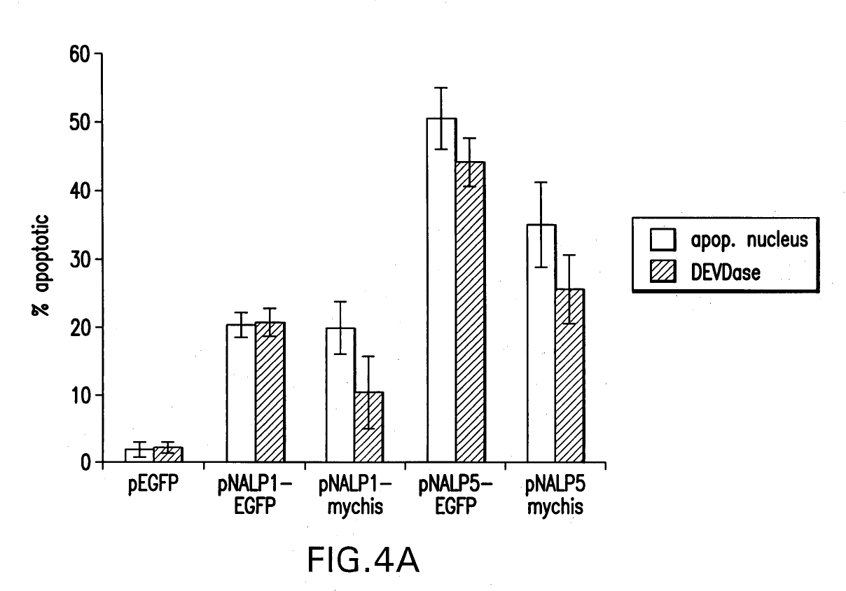

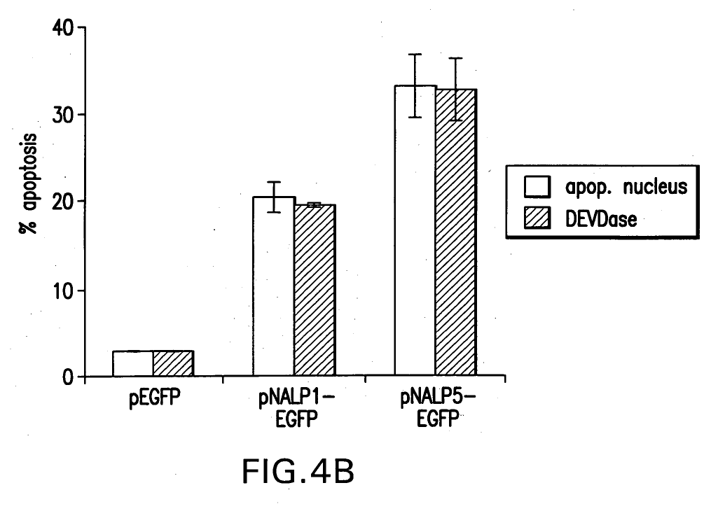

Recombinant expression assays were conducted, first in cell lines and then in cultured neurons, to determine whether NALP1 and NALP5 induce apoptosis. Human cDNA sequences for both genes were isolated and cloned into vectors providing target protein expression with fusions to either a myc-His6 epitope or enhanced green fluorescence protein (EGFP). NALP1-myc-His6 or NALP1-EGFP was expressed in several cell lines and found to induce cell death in some but not others. For example, HeLa and NIH-3T3 cells responded to the over-expression of NALP1 protein with morphological changes characteristic of apoptosis, whereas COS-7 or HEK-293 cells exhibited little or no response (data not shown). NALP5-myc-His6 and NALP5-EGFP showed a similar pattern of cell selectivity, with potent induction of apoptosis in HeLa or NIH-3T3 cells. Images of NALP1-EGFP or NALP5-EGFP expression in HeLa (data not shown) and the correlative activation of caspase-3, visualized by addition of a fluorogenic substrate (rhodamine-DEVD-FMK), are shown in FIG. 4A. Programmed cell death induced by NALP protein expression in HeLa was scored by nuclear morphology and activation of fluorogenic caspase-3 substrate. NALP1 and NALP5 proteins induced a significant elevation in apoptosis by both measures. NALP5 expressing cells consistently showed a greater propensity to undergo apoptosis than those expressing NALP1. These demonstrations that NALP1 and NALP5 can induce apoptosis in cell lines provide evidence for involvement of the proteins in cell death signaling.

To determine whether NALP1 and NALP5 could also induce apoptosis in neurons, rat CGNs were transfected with pEGFP vector, pNALP1-EGFP or pNALP5-EGFP. Transfected (EGFP+) neurons were scored for apoptosis by caspase-3 (DEVDase) activity or nuclear morphology. Elevated expression of NALP1 or NALP5 resulted in a highly significant increase in apoptosis, with both measures providing equivalent results (FIG. 4B). Recombinant expression of both NALP1 and NALP5 induced neuronal apoptosis, suggesting that the elevated gene expression observed in injured neurons (FIG. 2) or in MCAO samples (FIG. 3) represents the normal regulation of the functions of these molecules in neuronal death.

### Example 5

### Expression Knockdown of Native NALP1 Protects HeLa Cells from Apoptotic Insult

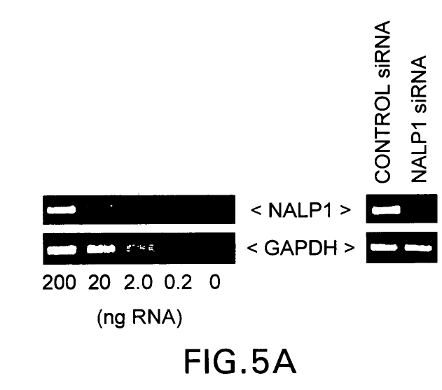

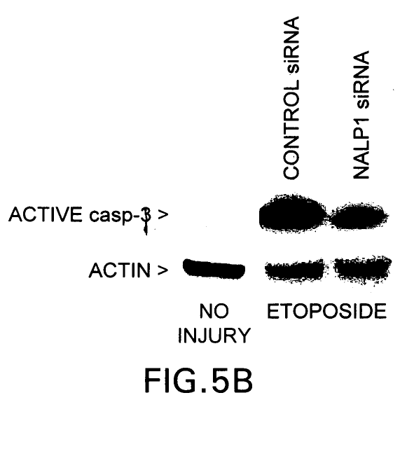

The ability of recombinantly expressed NALP1 to kill cells prompted the important question of whether it could be demonstrated by an expression knockdown approach that endogenous NALP1 is involved in apoptosis regulation. NALP1 mRNA is present in HeLa cells but at levels more than 10-fold lower than that of GAPDH mRNA (FIG. 5A). To test whether knockdown of NALP1 expression would protect HeLa from apoptosis, specific siRNA oligonucleotides for human NALP1 were generated. HeLa cells were transfected with NALP1 siRNA or control siRNA (i.e., scrambled sequence), and total cellular RNA was isolated 48 hours after transfection. In parallel, cells were treated with 300 nM etoposide to induce apoptosis. RT-PCR analysis indicated that mRNA of NALP1 was substantially reduced in cells transfected with the NALP1 siRNA compared to cells transfected with control siRNA (FIG. 5A). Treatment with apoptosis inducers, such as etoposide, resulted in activation of caspase-3, revealed by detection with a specific antibody for a neo-epitope on the active enzyme (FIG. 5B). Similar results were generated with serum withdrawal challenge (data not shown), establishing the generality of the protective effect of NALP1 knockdown. These data are consistent with the inverse outcome of apoptosis stimulation upon over-expression of NALP1, and suggest an important function for NALP1 in apoptosis regulation.

### Example 6

### NALP1 Binds dATP Through its Predicted Nucleotide Binding Site

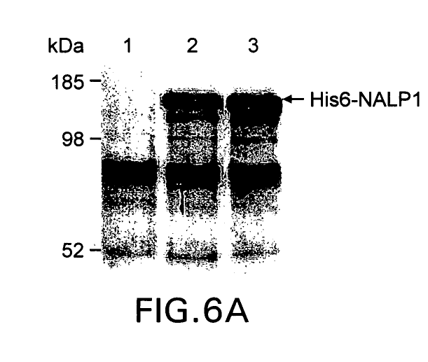

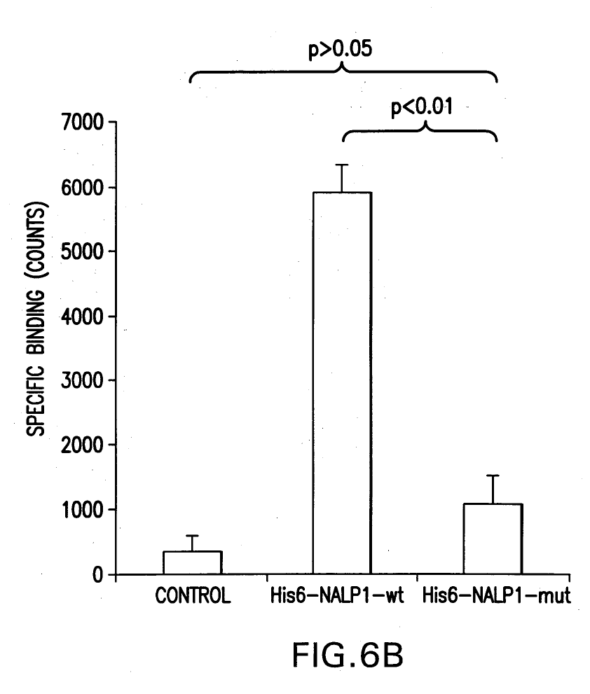

Examination of the NALP1 protein sequence reveals a well-defined nucleotide binding sequence (NBS) (Hliange et al., 2001; Chu et al., 2001), similar to mammalian Ced-4 homologues such as Apaf-1 and Nod1. The NBS of NALP1 includes consensus P-loop and Mg²⁺ binding motifs which contain the conserved amino acids as described by Walker et al., (1982). The NBS domain of Apaf-1 is critical for its function. Apaf-1 has been shown to bind nucleotide dATP, thereby stimulating the formation of a cytochrome c/Apaf-1/Caspase-9 complex and the consequent initiation of an apoptotic protease cascade (Li et al., 1997; Liu et al., 1996; Cain et al., 1999). Investigations of the purine nucleotide binding function of Apaf-1 has shown that dATP has the greatest affinity (Jiang et al., 2000). To test whether NALP1 binds to dATP, amino acid substitutions were generated within the NBS to provide a negative control. Specifically, the highly conserved glycine residue 339 and lysine residue 340 of SEQ ID NO:2, in the P-loop, were replaced by glutamic acid and alanine, respectively. Wild-type or NBS mutant His6-tagged NALP1 was transiently expressed in COS-7 cells and purified from cell lysates by Ni⁺ chromotography (FIG. 6A). Purified protein fractions were investigated for dATP binding using scintillation proximity (flashplate) methods. Specific binding was determined by measuring the amount of dATPa³⁵S displaced by non-radiolabeled dATP. Wide-type NALP1 showed specific binding of dATP, whereas NBS mutant NALP1 failed to achieve significant binding (FIG. 6B).

### Example 7

### Mutation of the NBS in NALP1 Diminishes Caspase-3 Activation

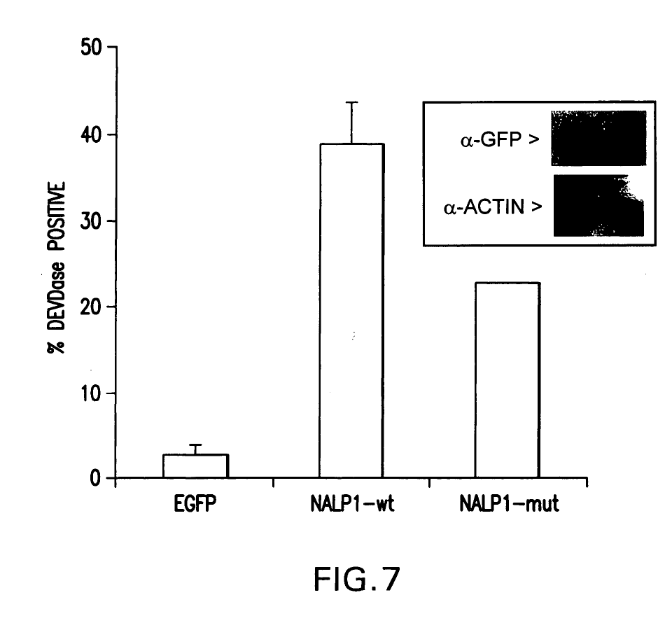

Wild-type or NBS mutant NALP1 was expressed in HeLa cells to investigate the role of nucleotide binding in NALP1-dependent caspase-3 activation and apoptosis. Cells were scored 48 hours post-transfection by cleavage of fluorogenic caspase-3 substrate (FIG. 7) and by inspection of nuclear and cellular morphology (data not shown). Mutation of the NBS resulted in a 50% reduction in the frequency of apoptotic cells versus wild-type NALP1. These data strongly suggest that nucleotide binding is important in achieving complete NALP1 apoptotic activity.

### Example 8

### Stimulation of NALP1 Activity by Addition of dATP

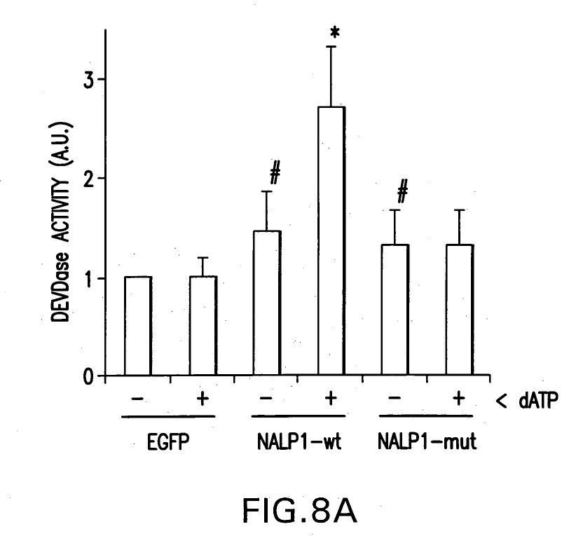

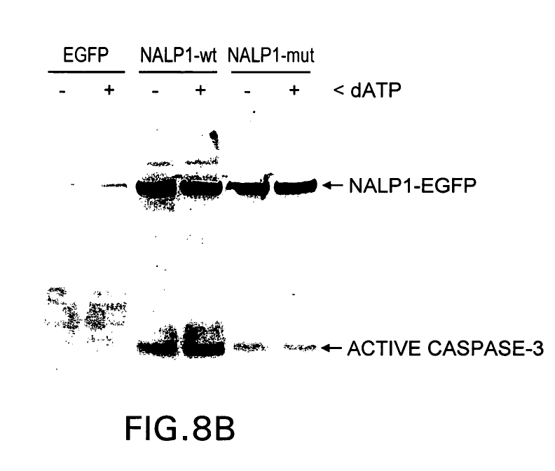

Caspases can be activated in vitro by incubating cell extracts with dATP. To further characterize the role of dATP in modulating NALP1 pro-apoptotic activity, a cell-free system was adapted from previously described assays (Jiang et al., 2000). Wild-type or NBS mutant NALP1 was transiently expressed in HeLa cells. Cells were homogenized 24 hours after transfection and soluble extract was prepared. After 1 hour incubation of cell extracts with addition of dATP and MgCl₂, fluorogenic substrate Ac-DEVD-AMC was added and caspase-3 activity was measured by spectrofluorometry. Parallel extracts were analyzed by immunoblotting with anti-GFP or anti-activated caspase-3 antibodies. In the absence of dATP, cells expressing wild-type NALP1 or NBS mutant had slightly elevated DEVDase activity compared to vector alone (FIG. 8A). Upon incubation with dATP, cells expressing wild-type NALP1 exhibited significantly elevated DEVDase activity, whereas extracts containing NBS mutant NALP1 did not respond to dATP addition, further demonstrating an important function for nucleotide binding in NALP1 regulation of apoptotic caspase cascades. Similar results were obtained using immunochemical methods to assess caspase-3 activation (FIG. 8B). Again, these data indicate that dATP binding to NALP1 protein is necessary for full activation of the NALP1-associated apoptotic caspase cascade.

Equivalents: Those skilled in the art will recognize, or be able to ascertain using no more than routine experimentation, many equivalents to the specific embodiments of the invention described herein. Such equivalents are intended to be encompassed by the following claims.

### REFERENCES

European Application No. EP 125023

European Application No. EP 171496

European Application No. EP 184187

U.S. Pat. No. 4,522,811

U.S. Pat. No. 4,554,101

U.S. Pat. No. 4,683,202

U.S. Pat. No. 4,816,567

U.S. Pat. No. 4,987,071

U.S. Pat. No. 5,116,742

U.S. Pat. No. 5,223,409

U.S. Pat. No. 5,328,470

U.S. Pat. No. 5,817,879

U.S. Pat. No. 5,933,819

U.S. Pat. No. 6,054,297

U.S. Pat. No. 6,420,119

International Application No. WO 86/01533

International Application No. WO 90/02809

International Application No. WO 91/17271

International Application No. WO 92/01047

International Application No. WO 92/09690

International Application No. WO 92/15679

International Application No. WO 92/18619

International Application No. WO 92/20791

International Application No. WO 93/01288

International Application No. WO 00/63364

Acehan et al., “Three-dimensional structure of the apoptosome: implications for assembly, procaspase-9 binding, and activation,” Mol. Cell. 9: 423-432, 2002.

Altschul et al., “Basic local alignment search tool,” J. Mol. Biol. 215: 403-410,1990.

Amann et al., Gene 69: 301-315,1988.

Anderson et al., “DNA Damage and Apoptosis in Alzheimer's Disease: Colocalization with c-Jun Immunoreactivity, Relationship to Brain Area, and Effect of Postmortem Delay,” J. Neurosci. 16(5): 1710-1719,1996.

Anderson, “Techniques for the preservation of three-dimensional structure in preparing specimens for the electron microscope.” Trans. N.Y. Acad. Sci. 13(130): 130-134,1951.

Baldari et al., Embo J. 6: 229-234,1987.

Barany et al., Int. J. Peptide Protein Res., 30: 705-739, 1987.

Bartel and Szostak, Science 261: 1411-1418,1993.

Belayev et al., “Middle cerebral artery occlusion in the rat by intraluminal suture: neurological and pathological evaluation of an improved model,” Stroke 27: 1616-1623,1996.

Bertin and DiStefano “The PYRIN domain: a novel motif found in apoptosis and inflammation proteins,” Cell Death Differ. 7(12): 1273-1274, 2000.

Boatright et al., “A unified model for apical caspase activation,” Mol. Cell 11: 529-541, 2003.

Bossenmeyer-Pourie et al., “CPP32/Caspase-3-like proteases in hypoxia-induced apoptosis in developing brain neurons,” Mol Brain Res 71: 225-237,1999.

Brazil et al., “Model Peptide Studies Demonstrate That Amphipathic Secondary Structures Can Be Recognized by the Chaperonin GroEL (Cpn60),” J. Biol. Chem., 272: 5105-5111,1997.

Cain et al., J. Biol. Chem. 274: 22686-22692, 1999.

Chen et al., “Induction of caspase-3-like protease may mediate delayed neuronal death in the hippocampus after transient cerebral ischemia,” J Neurosci 18(13): 4914-4928,1998.

Chiang et al., “An orchestrated gene expression component of neuronal programmed cell death revealed by cDNA array analysis,” Proc Natl Acad Sci USA 98(5): 2814-2819, 2001.

Chu et al., “A novel enhancer of the Apaf1 apoptosome involved in cytochrome c-dependent caspase activation and apoptosis,” J Biol Chem 276(12): 9239-9245, 2001. Cryan, Markou and Lucki, “Assessing antidepressant activity in rodents: recent developments and future needs,” TRENDS in Pharmacological Sciences 23(5), 2002.

Elbashir et al., Methods, 26: 199-213, 2003.

Frohman et al., Proc. Natl. Acad. Sci. USA 85: 8998-9002,1988.

Gaultier et al., Nucleic Acids Res. 15: 6625-6641,1987.

Gentz et al., Proc. Natl. Acad. Sci. USA, 86: 821-824,1989.

Gorman et al., “Apoptosis in neuronal cells: role of caspases,” NeuroRep 9(10): R49-R55,1998.

Gould et al., “Learning enhances adult neurogenesis in the hippocampal formation,” Nature Neuroscience, 2(3): 260-5,1999.

Elbashir et al., “Analysis of gene function in somatic mammalian cells using small interfering RNAs,” Methods 26,199-213, 2003.

Harper et al., Cell, 75: 805-816, 1993.

Hartmann et al., “Caspase-3: A vulnerability factor and final effector in apoptotic death of dopaminergic neurons in Parkinson's disease,” Proc Natl Acad Sci U S A 97(6): 2875-80, 2000.

Haselhoff and Gerlach, Nature 334: 585-591,1988.

Helene et al., Ann. N.Y. Acad Sci. 660: 27-36,1992.

Helene, Anticancer Drug Des. 6(6): 569-84, 1991.

Hlaing et al., “Molecular cloning and characterization of DEFCAP-L and —S, two isoforms of a novel member of the mammalian Ced-4 family of apoptosis proteins,” J Biol Chem 276(12): 9230-9238, 2001. Hofmann et al., “The CARD domain: a new apoptotic signalling motif,” Trend Biochem Sci22: 155-156,1997.

Inoue et al., FEBS Lett. 215: 327-330,1987(a).

Inoue et al., Nucleic Acids Res. 15: 6131-6148,1987(b).

Jiang and Wang, J. Biol. Chem. 275: 31199-31203, 2000

Johnson et al., Endoc. Rev., 10: 317-331,1989.

Kajkowski et al., “B-amyloid peptide-induced apoptosis regulated by a novel protein containing a G protein activation module,” J Biol Chem 276(22): 18748-18756,2001.

Kaufman et al., EMBO J. 6: 187-195,1987.

Kinouchi et al., “Induction of 70-kDa heat shock protein and hsp70 mRNA following transient focal cerebral ischemia in the rat,” J Cereb BI Flow Metabol 13: 105-115,1993.

Koonin and Aravind, “The NACHT family—a new group of predicted NTPases implicated in apoptosis and MHC transcription activation,” Trend Biol Sci 25: 223-224, 2000.

Kurjan and Herskowitz, Cell 933-943, 1982.

Kyte and Doolittle, J. Mol. Biol., 157:105-132,1982.

LeBlanc, Apoptosis and Alzheimer's disease. Molecular Mechanisms of Dementia. W. W. a. R. E. Tanzi. Totowa, N.J., Humana Press: 57-71,1996.

Li et al., “Induction of DNA fragmentation after 10 to 120 minutes of focal ischemia in rats,” Stroke 26: 1252-1258, 1995.

Li et al., Cell 91: 479-489, 1997.

Liu et al., Cell 86: 147-157,1996.

Longa et al., “Reversible middle cerebral artery occlusion without craniectomy in rats,” Stroke 20: 84-91,1989.

Maher, Bioassays 14(12): 807-15,1992.

Martinon et al., “The inflammasome: a molecular platform triggering activation of inflammatory caspases and processing of prolL-b,” Mol Cell 10: 417-426, 2002.

Martinon et al., “The pyrin domain: a possible member of the death domain-fold family implicated in apoptosis and inflammation,” Curr Biol 10: R118-R120, 2001.

Masumoto et al., “ASC, a novel 22-kDa protein, aggregates during apoptosis of human promyelocytic leukemia HL-60 cells,” J Biol Chem 274(48): 33835-33838,1999.

Miller and Johnson, “Metabolic and genetic analyses of apoptosis in potassium/serum-deprived rat cerebellar granule cells,” J Neurosci 16(23): 7487-7495,1996.

Namura et al., “Activation and cleavage of caspase-3 in apoptosis induced by experimental cerebral ischemia,” J Neurosci 18(10): 3659-3668,1998.

Needleman and Wunsch, “A general method applicable to the search for similarities in the amino acid sequence of two proteins,” J. Mol. Biol. 48(3): 443-453, 1970.

Pathan et al., “TUCAN, an antiapoptotic caspase-associated recruitment domain family protein overexpressed in cancer,” J Biol Chem 276(34): 32220-32229, 2001.

Pawlowski et al., “PAAD—a new protein domain associated with apoptosis, cancer and autoimmune diseases,” Trend Biochem Sci 26(2): 85-87, 2001.

Razmara et al., “CARD-8 protein, a new CARD family member that regulates caspase-1 activation and apoptosis,” J Biol Chem 277(16): 13952-13958, 2002.

Sambrook et al., “Molecular Cloning: A Laboratory Manual” 2nd, ed, Cold Spring Harbor Laboratory, Cold Spring Harbor Laboratory Press, Cold Spring Harbor, N.Y., 1989.

Schultz et al., Gene 54: 113-123,1987.

Seed, Nature 329: 840,1987.

Smith and Johnson, Gene 67: 31-40,1988.

Studier et al. “Gene Expression Technology” Methods in Enzymology 185: 60-89, 1990

Thompson, Science, 267:1456-1462,1995.

Thorner et al., “Applications of Chimeric Genes and Hybrid Proteins, Part A, Gene Expression and Protein Purification,” Methods in Enzymology, Vol:326, Academic Press, 2000(a).

Thorner et al., “Applications of Chimeric Genes and Hybrid Proteins, Part B, Cell Biology and Physiology,” Methods in Enzymology, Vol:326, Academic Press, 2000(b).

Thorner et al., “Applications of Chimeric Genes and Hybrid Proteins, Part C, Protein-Protein Interactions and Genomics,” Methods in Enzymology, Vol:326, Academic Press, 2000(c).

Tong et al., “A human homologue of mouse MATER, a maternal effect gene essential for early embryonic development,” Hum Reprod 17(4): 903-911, 2002.

Troncoso et al., “In situ labeling of dying cortical neurons in normal aging and in Alzheimer's disease: correlations with senile plaques and disease progression,” J Neuropathol Exp Med 55: 1134-1142,1996.

Tschopp et al., “NALPs: A novel protein family involved in inflammation,” Nat Rev Mol Cell Biol 4: 95-104, 2003.

Walker et al., EMBO J. 1: 945-951,1982.

Xia et al., “Calcium influx via the NMDA receptor induces immediate early gene transcription by a MAP kinase/ERK-dependent mechanism,” J Neurosci 16(17): 5425-5436,1996.

Yuan and Yankner, “Apoptosis in the nervous system,” Nature 407: 802-809, 2000.

Zou et al., “An APAF-1-cytochrome c multimeric complex is a functional apoptosome that activates procaspase-9,” J Biol Chem 274(17): 11549-11556, 1999

What is claimed is:

1. A method for screening compounds which inhibit NALP polypeptide activity comprising the steps of:
(a) providing a host cell comprising a polynucleotide expressing a NALP1 polypeptide;
(b) contacting the cell with a test compound; and
(c) assaying caspase activity,
wherein a decrease in caspase activity indicates the test compound inhibits NALP1 activity.

2. The method of claim 1, wherein the host cell in step (a) further comprises a polynucleotide expressing a NALP5 polypeptide.

3. A method for screening compounds which inhibit NALP polypeptide activity comprising the steps of:
(a) providing a host cell comprising a polynucleotide expressing a NALP5 polypeptide;
(b) contacting the cell with a test compound; and
(c) assaying caspase activity,
wherein a decrease in caspase activity indicates the test compound inhibits NALP5 activity.

4. The method of claim 3, wherein the host cell in step (a) further comprises a polynucleotide expressing a NALP1 polypeptide.

5. The method according to claims 1 or 3, wherein the host cell is a mammalian cell.

6. The method of claim 5, wherein the mammalian cell is a HeLa cell or NIH-3T3 cell.

7. The method of claim 5, wherein the mammalian cell is a neuronal cell.

8. The method of claim 7, wherein the neuronal cell is a cerebellar granule neuron (CGN), a cortical neuron or a hippocampus neuron.

9. The method according to claims 1 or 4, wherein the NALP1 polypeptide is a fusion polypeptide.

10. The method of claim 9, wherein the NALP1 fusion polypeptide comprises an epitope tag.

11. The method of claim 10, wherein the NALP1 fusion polypeptide is a NALP1-myc-His fusion, wherein the myc-His polypeptide is at the carboxy terminus of the NALP1 polypeptide.

12. The method according to claims 2 or 3, wherein the NALP5 polypeptide is a fusion polypeptide.

13. The method of claim 12, wherein the NALP5 fusion polypeptide comprises an epitope tag.

14. The method of claim 13, wherein the NALP5 fusion polypeptide is a NALP5-myc-His fusion polypeptide, wherein the myc-His polypeptide is at the carboxy terminus of the NALP5 polypeptide.

15. The method according to claims 1 or 3, wherein assaying caspase activity comprises detecting a fluorescent caspase-3 substrate.

16. The method of claim 15, wherein the caspase-3 substrate is a fluorescent sulforhodamine-DEVD-FMK.

17. The method according to claims 1 or 3, wherein the test compound is selected from the group consisting of an organic molecule, a polypeptide, a peptide fragment, a peptide mimetic, an antisense RNA and a small interference RNA.

18. The method of claim 17, wherein the organic molecule is a nucleotide analogue.

19. The method of claim 18, wherein the nucleotide analogue is a purine.

20. The method according to claims 1 or 4, wherein the polynucleotide encoding the NALP1 polypeptide comprises a nucleic acid sequence of SEQ ID NO:1.

21. The method of claim 20, wherein the polynucleotide is comprised within a mammalian expression vector.

22. The method of claim 21, wherein the vector is a plasmid.

23. The method of claim 22, wherein the plasmid is selected from the group consisting of pcDNA3.1, pEGFP and pCMV.

24. The method of claim 20, wherein the polynucleotide is operatively linked to a promoter selected from the group consisting of CMV, ADH, TRE, LTR, TK and SV40.

25. The method of claim 2 or 3, wherein the polynucleotide encoding the NALP5 polypeptide comprises a nucleic acid sequence of SEQ ID NO:3.

26. The method of claim 25, wherein the polynucleotide is comprised within a mammalian expression vector.

27. The method of claim 26, wherein the vector is a plasmid.

28. The method of claim 27, wherein the vector is a plasmid selected from the group consisting of pcDNA3.1, pEGFP and pCMV.

29. The method of claim 25, wherein the polynucleotide is operatively linked to a promoter selected from the group consisting of CMV, ADH, TRE, LTR, TK and SV40.

30. A method for screening compounds which inhibit NALP polypeptide activity comprising the steps of:
(a) providing a host cell comprising a polynucleotide expressing a NALP1 polypeptide;
(b) contacting the cell with a test compound; and
(c) detecting cell morphology;
wherein no change in cell morphology indicates the compound inhibits NALP1 activity.

31. The method of claim 30, wherein the host cell in step (a) further comprises a polynucleotide expressing a NALP5 polypeptide.

32. A method for screening compounds which inhibit NALP polypeptide activity comprising the steps of:
(a) providing a host cell comprising a polynucleotide expressing a NALP5 polypeptide;
(b) contacting the cell with a test compound; and
(c) detecting cell morphology;
wherein no change in cell morphology indicates the compound inhibits NALP5 activity.

33. The method of claim 32, wherein the host cell in step (a) further comprises a polynucleotide expressing a NALP1 polypeptide.

34. The method according to claims 30 or 32, wherein the host cell is a mammalian cell.

35. The method of claim 34, wherein the mammalian cell is a HeLa cell or NIH-3T3 cell.

36. The method of claim 34, wherein the host cell is a neuronal cell.

37. The method of claim 36, wherein the neuronal cell is a CGN, a cortical neuron or a hippocampus neuron.

38. The method according to claims 30 or 33, wherein the NALP1 polypeptide is a fusion polypeptide.

39. The method of claim 39, wherein the fusion polypeptide is a NALP1-EGFP fusion polypeptide, wherein the EGFP is at the carboxy terminus of the NALP1 polypeptide.

40. The method according to claims 31 or 32, wherein the NALP5 polypeptide is a fusion polypeptide.

41. The method of claim 40, wherein the fusion polypeptide is a NALP5-EGFP fusion polypeptide, wherein the EGFP is at the carboxy terminus of the NALP5 polypeptide.

42. The method according to claims 30 or 32, wherein the test compound is selected from the group consisting of an organic molecule, a polypeptide, a peptide fragment, a peptide mimetic, an antisense RNA and a small interference RNA.

43. The method of claim 42, wherein the organic molecule is a nucleotide analogue.

44. The method of claim 43, wherein the nucleotide analogue is a purine.

45. The method according to claims 30 or 33, wherein the polynucleotide encoding the NALP1 polypeptide comprises a nucleic acid sequence of SEQ ID NO:1.

46. The method of claim 45, wherein the polynucleotide is comprised within a mammalian expression vector.

47. The method of claim 46, wherein the vector is a plasmid.

48. The method of claim 47, wherein the vector is a plasmid selected from the group consisting of pEGFP, pcDNA3.1 and pCMV.

49. The method of claim 45, wherein the polynucleotide is operatively linked to a promoter selected from the group consisting of CMV, ADH, TRE, LTR, TK and SV40.

50. The method of claim 31 or 32, wherein the polynucleotide encoding the NALP5 polypeptide comprises a nucleic acid sequence of SEQ ID NO:3.

51. The method of claim 50, wherein the polynucleotide is comprised within a mammalian expression vector.

52. The method of claim 51, wherein the vector is a plasmid.

53. The method of claim 52, wherein the vector is a plasmid selected from the group consisting of pEGFP, pcDNA3.1 and pCMV

54. The method of claim 50, wherein the polynucleotide is operatively linked to a promoter selected from the group consisting of CMV, ADH, TRE, LTR, TK and SV40.

55. A method for screening compounds which inhibit NALP polypeptide activity comprising the steps of:
(a) providing a host cell comprising a polynucleotide expressing a NALP1 polypeptide;
(b) contacting the cell with a test compound; and
(c) detecting nuclear morphology;
wherein no change in nuclear morphology indicates the compound inhibits NALP1 activity.

56. The method of claim 55, wherein the host cell in step (a) further comprises a polynucleotide expressing a NALP5 polypeptide.

57. A method for screening compounds which inhibit NALP polypeptide activity comprising the steps of:
(a) providing a host cell comprising a polynucleotide expressing a NALP5 polypeptide;
(b) contacting the cell with a test compound; and
(c) detecting cell morphology;
wherein no change in nuclear morphology indicates the compound inhibits NALP5 activity.

58. The method of claim 57, wherein the host cell in step (a) further comprises a polynucleotide expressing a NALP1 polypeptide.

59. A method for screening compounds which inhibit NALP polypeptide activity comprising the steps of:
(a) providing a host cell comprising a polynucleotide expressing a NALP1 polypeptide;
(b) contacting the cell with a test compound; and
(c) detecting cell viability;
wherein cell viability indicates the compound inhibits NALP1 activity.

60. The method of claim 59, wherein the host cell in step (a) further comprises a polynucleotide expressing a NALP5 polypeptide.

61. A method for screening compounds which inhibit NALP polypeptide activity comprising the steps of:
(a) providing a host cell comprising a polynucleotide expressing a NALP5 polypeptide;
(b) contacting the cell with a test compound; and
(c) detecting cell viability;
wherein cell viability indicates the compound inhibits NALP5 activity.

62. The method of claim 61, wherein the host cell in step (a) further comprises a polynucleotide expressing a NALP1 polypeptide.

63. A method for screening compounds which inhibit apoptosis in a mammalian cell comprising the steps of:
(a) providing a host cell comprising a polynucleotide expressing a NALP1 polypeptide;
(b) contacting the cell with a test compound; and
(c) assaying caspase activity,
wherein a decrease in caspase activity indicates the test compound inhibits apoptosis.

64. A method for screening compounds which inhibit apoptosis in a mammalian cell comprising the steps of:
(a) providing a host cell comprising a polynucleotide expressing a NALP1 polypeptide;
(b) contacting the cell with a test compound; and
(c) detecting cell morphology;
wherein no change in cell morphology indicates the compound inhibits apoptosis.

65. A method for screening compounds which inhibit apoptosis in a mammalian cell comprising the steps of:
(a) providing a host cell comprising a polynucleotide expressing a NALP1 polypeptide;
(b) contacting the cell with a test compound; and
(c) detecting nuclear morphology;
wherein no change in nuclear morphology indicates the compound inhibits apoptosis.

66. A method for screening compounds which inhibit apoptosis in a mammalian cell comprising the steps of:
(a) providing a host cell comprising a polynucleotide expressing a NALP1 polypeptide;
(b) contacting the cell with a test compound; and
(c) detecting cell viability;
wherein cell viability indicates the compound inhibits apoptosis.

67. A method for detecting neuron damage in a mammalian subject comprising the steps of:
(a) obtaining a biological sample from the subject;
(b) contacting the sample with a polynucleotide probe complementary to a NAPL1 mRNA or a NALP5 mRNA;
(c) measuring the amount of probe bound to the mRNA; and
(d) comparing the amount in step (c) with NALP1 mRNA or NALP5 mRNA in mammalian samples obtained from a statistically significant population lacking neuron damage,
wherein higher NALP1 or NALP5 levels in the subject indicates neuron damage.

68. A method for detecting neuron damage in a mammalian subject comprising the steps of:
(a) obtaining a biological sample from the subject;
(b) contacting the sample with a polynucleotide probe complementary to a NAPL1 mRNA and a polynucleotide probe complementary to a NALP5 mRNA;
(c) measuring the amount of each probe bound to the mRNA; and
(d) comparing the amount in step (c) with NALP1 mRNA and NALP5 mRNA in mammalian samples obtained from a statistically significant population lacking neuron damage,
wherein higher NALP1 or NALP5 levels in the subject indicates neuron damage.

69. The method according to claims 67 or 68, wherein the probe complementary to the NALP1 mRNA comprises a nucleotide sequence which hybridizes under high stringency hybridization conditions with a polynucleotide comprising the nucleotide sequence of SEQ ID NO:1.

70. The method according to claims 67 or 68, wherein the probe complementary to the NALP5 mRNA comprises a nucleotide sequence which hybridizes under high stringency hybridization conditions with a polynucleotide comprising the nucleotide sequence of SEQ ID NO:3.

71. The method according claims 67 or 68, wherein the biological sample is selected from the group consisting of blood plasma, serum, erythrocytes, leukocytes, platelets, lymphocytes, macrophages, fibroblast cells, mast cells, fat cells, epithelial cells, nerve cells, glial cells, Schwann cells, progenitor stem cells, a cerebrospinal fluid (CSF), saliva, a skin biopsy, a brain biopsy and a buccal biopsy.

72. The method according to claims 67 or 68, wherein the polynucleotide probe is labeled with a radioactive isotope or a fluorophore.

73. A method for measuring the expression levels of a NALP1 gene and NALP5 gene in a rat neuron comprising the steps of:
(a) obtaining a cultured rat neuron cell;
(b) isolating the total RNA from step (a);
(c) generating a NALP1 cDNA and a NALP5 cDNA from the RNA of step (b) by PCR using a 5′ NALP1 PCR primer comprising a nucleic acid sequence of SEQ ID NO:23, a 3′ NALP1 PCR primer comprising a nucleic acid sequence of SEQ ID NO:24; a 5′ NALP5 PCR primer comprising a nucleic acid sequence of SEQ ID NO:23 and a 3′ NALP5
PCR primer comprising a nucleic acid sequence of SEQ ID NO:24; and
(d) detecting the amount of the cDNA in step (c).

74. The method of claim 73, wherein the neuron cell is a CGN, a cortical neuron or a hippocampus neuron.

75. The method of claim 73, wherein the cDNA comprises a radioactive dNTP.

76. A method for assaying neuron damage or injury in a rat neuron cell comprising the steps of:
(a) obtaining a cultured rat neuron cell;
(b) injuring the cell by transfer to a culture medium having no serum and a reduced K⁺ concentration of about 5 mM;
(c) isolating the total RNA from step (b);
(d) generating a NALP1 cDNA and a NALP5 cDNA from the RNA of step (c) by PCR using a 5′ NALP1 PCR primer comprising a nucleic acid sequence of SEQ ID NO:23, a 3′ NALP1 PCR primer comprising a nucleic acid sequence of SEQ ID NO:24; a 5′ NALP5 PCR primer comprising a nucleic acid sequence of SEQ ID NO:23 and a 3′ NALP5 PCR primer comprising a nucleic acid sequence of SEQ ID NO:24;
(e) detecting the amount of the cDNA in step (d),
wherein an increase in either NALP1 or NALP5 cDNA in step (e), relative to a non-injured neuron control, indicates neuron injury or damage.

77. A method for monitoring the kinetics of neuron injury comprising the steps of:
(a) subjecting a population of adults rats to transient middle cerebral artery occlusion (MCAO) for about 1 hour and immediately reperfusing;
(b) obtaining at a desired kinetic time point a rat from step (a), wherein cortex tissue from the rat is dissected and frozen;
(c) repeating step (b) for each desired time point;
(d) isolating the total RNA from the tissue in each time point;
(e) generating a NALP1 cDNA and a NALP5 cDNA from the RNA of step (d) by PCR using a 5′ NALP1 PCR primer comprising a nucleic acid sequence of SEQ ID NO:23, a 3′ NALP1 PCR primer comprising a nucleic acid sequence of SEQ ID NO:24; a 5′ NALP5 PCR primer comprising a nucleic acid sequence of SEQ ID NO:23 and a 3′ NALP5 PCR primer comprising a nucleic acid sequence of SEQ ID NO:24;
(f) detecting the amount of the cDNA in step (e).

78. A method for screening compounds which inhibit the expression of a NALP1 polypeptide comprising the steps of:
(a) providing a host cell comprising a polynucleotide expressing a NALP1 polypeptide;
(b) contacting the cell with a test compound; and
(c) assaying NALP1 gene expression,
wherein a decrease in NALP1 gene expression indicates the test compound inhibits the NALP1 apoptosis pathway.

79. A method for screening compounds which inhibit the expression of a NALP5 polypeptide comprising the steps of:
(a) providing a host cell comprising a polynucleotide expressing a NALP5 polypeptide;
(b) contacting the cell with a test compound; and
(c) assaying NALP5 gene expression,
wherein a decrease in NALP5 gene expression indicates the test compound inhibits the NALP5 apoptosis pathway.

80. An antisense RNA molecule which inhibits the expression of a polynucleotide encoding a NALP1 polypeptide comprising an amino acid sequence of SEQ ID NO:2.

81. The RNA molecule of claim 80, wherein the molecule is antisense to a polynucleotide having a nucleotide sequence of SEQ ID NO:1 or a degenerate variant thereof.

82. The RNA molecule of claim 81, wherein the molecule comprises a nucleotide sequence of SEQ ID NO:5.

83. An antisense RNA molecule which inhibits the expression of a polynucleotide encoding a NALP5 polypeptide comprising an amino acid sequence of SEQ ID NO:4.

84. The RNA molecule of claim 83, wherein the molecule is antisense to a polynucleotide having a nucleotide sequence of SEQ ID NO:3 or a degenerate variant thereof.

85. A method for inhibiting apoptosis in a cell comprising administering to the cell an expression construct comprising an RNA molecule according to any one of claims 80-84.

86. A polynucleotide encoding a mutated NALP1 polypeptide comprising an amino acid sequence of SEQ ID NO:2, wherein the glycine amino acid at position 339 is mutated to a glutamate amino acid.

87. A polypeptide comprising an amino acid sequence of SEQ ID NO:2, wherein the glycine amino acid at position 339 is mutated to a glutamate amino acid.

88. A polynucleotide encoding a mutated NALP1 polypeptide comprising an amino acid sequence of SEQ ID NO:2, wherein the lysine amino acid at position 340 is mutated to an alanine amino acid.

89. A polypeptide comprising an amino acid sequence of SEQ ID NO:2, wherein the lysine amino acid at position 340 is mutated to an alanine amino acid.

90. A polynucleotide encoding a mutated NALP1 polypeptide comprising an amino acid sequence of SEQ ID NO:2, wherein the glycine amino acid at position 339 is mutated to a glutamate amino acid and the lysine amino acid at position 340 is mutated to an alanine amino acid.

91. A polypeptide comprising an amino acid sequence of SEQ ID NO:2, wherein the glycine amino acid at position 339 is mutated to a glutamate amino acid and the lysine at amino acid position 340 is mutated to an alanine amino acid.

92. A polynucleotide encoding a NALP1 polypeptide of SEQ ID NO:2, wherein the amino acid sequence of SEQ ID NO:2 comprises a mutation in the nucleotide binding sequence (NBS) from amino acid 328 to amino acid 637.

93. A polypeptide comprising an amino acid sequence of SEQ ID NO:2, wherein the amino acid sequence of SEQ ID NO:2 comprises a mutation in the NBS from amino acid 328 to amino acid 637.

94. The polynucleotide of claim 92, wherein a mutation in the NBS is further defined as a mutation in the Mg²⁺ binding sequence of SEQ ID NO:2 comprising amino acid 392 through amino acid 415.

95. The polynucleotide of claim 94, wherein a mutation in the Mg²⁺ binding sequence of SEQ ID NO:2 is a mutation at an amino acid residue selected from the group consisting of glutamate 403 (Glu 403), aspartate 410 (Asp 410), aspartate 413 (Asp 413) and glutamate 414 (Glu 414)

96. The polypeptide of claim 93, wherein the mutation NBS is further defined as a mutation in the Mg²⁺ binding sequence of SEQ ID NO:2 comprising amino acid 392 through amino acid 415.

97. The polypeptide of claim 96, wherein a mutation in the Mg²⁺ binding sequence of SEQ ID NO:2 is a mutation at an amino acid residue selected from the group consisting of Glu 403, Asp 410, Asp 413 and Glu 414.

98. The polynucleotide according to one of claims 86, 88, 90, 92, 94 or 95, wherein the NALP1 polypeptide does not bind a purine nucleotide.

99. The polynucleotide of claim 98, wherein the purine is dATP.

100. The polypeptide according to one of claims 87, 89, 91, 93, 96 or 97, wherein the NALP1 polypeptide does not bind a purine nucleotide.

101. The polypeptide of claim 100, wherein the purine is dATP.

102. A method for screening compounds which activate a NALP1 polypeptide comprising the steps of:
(a) providing a host cell comprising a polynucleotide encoding a NALP1 polypeptide having a mutation in the NBS;
(b) contacting the cell with a test compound; and
(c) assaying NALP1 activity,
wherein an increase in NALP1 activity indicates the compound activates the polypeptide.

103. The method of claim 102, wherein the test compound is a nucleotide analogue of GTP, dGTP, ATP or dATP.

104. A polynucleotide encoding a NALP5 polypeptide of SEQ ID NO:4, wherein the amino acid sequence of SEQ ID NO:4 comprises a mutation in the nucleotide binding sequence (NBS) from amino acid 191 to amino acid 510.

105. A polypeptide comprising an amino acid sequence of SEQ ID NO:4, wherein the amino acid sequence of SEQ ID NO:4 comprises a mutation in the NBS from amino acid 191 to amino acid 510.

106. The polynucleotide of claim 104, wherein a mutation in the NBS is further defined as a mutation in the Mg²⁺ binding sequence of SEQ ID NO:4 comprising amino acid 357 through amino acid 367.

107. The polynucleotide of claim 106, wherein a mutation in the Mg²⁺ binding sequence of SEQ ID NO:4 is a mutation at an amino acid residue selected from the group consisting of Asp 362, Asp 365 and Asp 366.

108. The polypeptide of claim 105, wherein the mutation NBS is further defined as a mutation in the Mg²⁺ binding sequence of SEQ ID NO:2 comprising amino acid 357 through amino acid 367.

109. The polypeptide of claim 108, wherein a mutation in the Mg²⁺ binding sequence of SEQ ID NO:2 is a mutation at an amino acid residue selected from the group consisting of Asp 362, Asp 365 and Asp 366.

110. The polynucleotide according to one of claims 104, 106 or 107, wherein the NALP5 polypeptide does not bind a purine nucleotide.

111. The polynucleotide of claim 11, wherein the purine is dATP.

112. The polypeptide according to one of claims 105, 108 or 109, wherein the NALP5 polypeptide does not bind a purine nucleotide.

113. The polypeptide of claim 112, wherein the purine is dATP.

114. A method for screening compounds which activate a NALP5 polypeptide comprising the steps of:
(a) providing a host cell comprising a polynucleotide encoding a NALP5 polypeptide having a mutation in the NBS;
(b) contacting the cell with a test compound; and
(c) assaying NALP5 activity,
wherein an increase in NALP5 activity indicates the compound activates the polypeptide.

115. The method of claim 114, wherein the test compound is a nucleotide analogue of dGTP, GTP, ATP or dATP.

116. A pharmaceutical composition comprising a compound identified according to the methods of any one of claims 1, 3, 30, 32, 55, 57, 59, 61, 63, 64, 65, 66, 78, 79 102 or 114.


SEQUENCE LISTING (31 sequences)

SEQ ID NO: 1 - DNA, 5068 bp, Homo sapiens
  CDS (1)..(4422)
atggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactccaaaaaaaaaaa

SEQ ID NO: 2 - protein, 1473 aa, Homo sapiens
MAGGAWGRLACYLEFLKKEELKEFQLLLANKAHSRSSSGETPAQPEKTSGMEVASYLVAQYGEQRAWDLALHTWEQMGLRSLCAQAQEGAGHSPSFPYSPSEPHLGSPSQPTSTAVLMPWIHELPAGCTQGSERRVLRQLPDTSGRRWREISASLLYQALPSSPDHESPSQESPNAPTSTAVLGSWGSPPQPSLAPREQEAPGTQWPLDETSGIYYTEIREREREKSEKGRPPWAAVVGTPPQAHTSLQPHHHPWEPSVRESLCSTWPWKNEDFNQKFTQLLLLQRPHPRSQDPLVKRSWPDYVEENRGHLIEIRDLFGPGLDTQEPRIVILQGAAGIGKSTLARQVKEAWGRGQLYGDRFQHVFYFSCRELAQSKVVSLAELIGKDGTATPAPIRQILSRPERLLFILDGVDEPGWVLQEPSSELCLHWSQPQPADALLGSLLGKTILPEASFLITARTTALQNLIPSLEQARWVEVLGFSESSRKEYFYRYFTDERQAIRAFRLVKSNKELWALCLVPWVSWLACTCLMQQMKRKEKLTLTSKTTTTLCLHYLAQALQAQPLGPQLRDLCSLAAEGIWQKKTLFSPDDLRKHGLDGAIISTFLKMGILQEHPIPLSYSFIHLCFQEFFAAMSYVLEDEKGRGKHSNCIIDLEKTLEAYGIHGLFGASTTRFLLGLLSDEGEREMENIFHCRLSQGRNLMQWVPSLQLLLQPHSLESLHCLYETRNKTFLTQVMAHFEEMGMCVETDMELLVCTFCIKFSRHVKKLQLIEGRQHRSTWSPTMVVLFRWVPVTDAYWQILFSVLKVTRNLKELDLSGNSLSHSAVKSLCKTLRRPRCLLETLRLAGCGLTAEDCKDLAFGLRANQTLTELDLSFNVLTDAGAKHLCQRLRQPSCKLQRLQLVSCGLTSDCCQDLASVLSASPSLKELDLQQNNLDDVGVRLLCEGLRHPACKLIRLGLDQTTLSDEMRQELRALEQEKPQLLIFSRRKPSVMTPTEGLDTGEMSNSTSSLKRQRLGSERAASHVAQANLKLLDVSKIFPIAEIAEESSPEVVPVELLCVPSPASQGDLHTKPLGTDDDFWGPTGPVATEVVDKEKNLYRVHFPVAGSYRWPNTGLCFVMREAVTVEIEFCVWDQFLGEINPQHSWMVAGPLLDIKAEPGAVEAVHLPHFVALQGGHVDTSLFQMAHFKEEGMLLEKPARVELHHIVLENPSFSPLGVLLKMIHNALRFIPVTSVVLLYHRVHPEEVTFHLYLIPSDCSIRKAIDDLEMKFQFVRIHKPPPLTPLYMGCRYTVSGSGSGMLEILPKELELCYRSPGEDQLFSEFYVGHLGSGIRLQVKDKKDETLVWEALVKPGDLMPATTLIPPARIAVPSPLDAPQLLHFVDQYREQLIARVTSVEVVLDKLHGQVLSQEQYERVLAENTRPSQMRKLFSLSQSWDRKCKDGLYQALKETHPHLIMELWEKGSKKGLLPLSS

SEQ ID NO: 3 - DNA, 3885 bp, Homo sapiens
  CDS (1)..(3603)
atgaaggttgcaggaggacttgaacttggagctgctgctctgctctcagcatcaccacgtgctcttgtcactctttccacaggtcctacttgctctatattaccaaagaatccacttttcccccaaaacctgagctctcagccttgtatcaagatggaaggagacaaatcgctcaccttttccagctacgggctgcaatggtgtctctatgagctagacaaggaagaatttcagacattcaaggaattactaaagaagaaatcttcagaatcgaccacatgctctattccacagtttgaaatcgagaatgccaacgtggaatgtctggcactcctcttgcatgagtattatggagcatcgctggcctgggctacgtccattagcatctttgaaaacatgaacctgcgaaccctctcggagaaggcacgggatgacatgaaaagacattcaccagaagatcctgaagcaacgatgactgaccaaggaccaagcaaggaaaaagtgccaggaatttcacaagctgtgcaacaagatagtgccacagctgcagagacaaaagaacaggaaatttcacaagctatggaacaagaaggtgccacagcagcagagacagaagaacaagaaatttcacaagctatggaacaagaaggtgccacagcagcagagacagaagaacaaggacatggaggtgacacatgggactacaagagtcacgtgatgaccaaattcgctgaggaggaggatgtacgtcgtagttttgaaaacactgctgctgactggccggaaatgcaaacgttggctggtgcttttgattcagaccggtggggcttccggcctcgcacggtggttctgcacggaaagtcaggaattgggaaatcggctctagccagaaggatcgtgctgtgctgggcgcaaggtggactctaccagggaatgttctcctacgtcttcttcctccccgttagagagatgcagcggaagaaggagagcagtgtcacagagttcatctccagggagtggccagactcccaggctccggtgacggagatcatgtcccgaccagaaaggctgttgttcatcattgacggtttcgatgacctgggctctgtcctcaacaatgacacaaagctctgcaaagactgggctgagaagcagcctccgttcaccctcatacgcagtctgctgaggaaggtcctgctccctgagtccttcctgatcgtcaccgtcagagacgtgggcacagagaagctcaagtcagaggtcgtgtctccccgttacctgttagttagaggaatctccggggaacaaagaatccacttgctccttgagcgcgggattggtgagcatcagaagacacaagggttgcgtgcgatcatcaacaaccgtgagctgctcgaccagtgccaggtgcccgccgtgggctctctcatctgcgtggccctgcagctgcaggacgtggtgggggagagcgtcgcccccttcaaccaaacgctcacaggcctgcacgccgcttttgcgtttcatcagctcacccctcgaggcgtggtccggcgctgtctcaatctggaggaaagagttgtcctgaagcgcttctgccgtatggctgtggagggagtgtggaataggaagtcagtgtttgatggtgacgacctcatggttcaaggactcggggagtctgagctccgtgctctgtttcacatgaacatccttctcccagacagccactgtgaggagtactacaccttcttccacctcagtctccaggacttctgtgccgccttgtactacgtgttagagggcctggaaatcgagccagctctctgccctctgtacgttgagaagacaaagaggtccatggagcttaaacaggcaggcttccatatccactcgctttggatgaagcgtttcttgtttggcctcgtgagcgaagacgtaaggaggccactggaggtcctgctgggctgtcccgttcccctgggggtgaagcagaagcttctgcactgggtctctctgttgggtcagcagcctaatgccaccaccccaggagacaccctggacgccttccactgtcttttcgagactcaagacaaagagtttgttcgcttggcattaaacagcttccaagaagtgtggcttccgattaaccagaacctggacttgatagcatcttccttctgcctccagcactgtccgtatttgcggaaaattcgggtggatgtcaaagggatcttcccaagagatgagtccgctgaggcatgtcctgtggtccctctatggatgcgggataagaccctcattgaggagcagtgggaagatttctgctccatgcttggcacccacccacacctgcggcagctggacctgggcagcagcatcctgacagagcgggccatgaagaccctgtgtgccaagctgaggcatcccacctgcaagatacagaccctgatgtttagaaatgcacagattacccctggtgtgcaacacctctggagaatcgtcatggccaaccgtaacctaagatccctcaacttgggaggcacccacctgaaggaagaggatgtaaggatggcgtgtgaagccttaaaacacccaaaatgtttgttggagtctttgaggctggattgctgtggattgacccatgcctgttacctgaagatctcccaaatccttacgacctcccccagcctgaaatctctgagcctggcaggaaacaaggtgacagaccagggagtaacgcctctcagtgatgccttgagggtctcccagtgcgccctgcagaagctgatactggaggactgtggcatcacagccacgggttgccagagtctggcctcagccctcgtcagcaaccggagcttgacacacctgtgcctatccaacaacagcctggggaacgaaggtgtaaatctactgtgtcgatccatgaggcttccccactgtagtctgcagaggctgatgctgaatcagtgccacctggacacggctggctgtggttctcttgcacttgcgcttatgggtaactcatggctgacgcacctgagccttagcatgaaccctgtggaagacaatggcgtgaagcttctgtgcgaggtcatgagagaaccatcttgtcatctccaggacctggagttggtaaagtgtcatctcaccgccgcgtgctgtgagagtctgtcctgtgtgatctcgaggagcagacacctgaagagcctggatctcacggacaatgccctgggtgacggtggggttgctgcgctgtgcgagggactgaagcaaaagaacagtgttctgacgagactcgggttgaaggcatgtggactgacttctgattgctgtgaggcactctccttggccctttcctgcaaccggcatctgaccagtctaaacctggtgcagaataacttcagtcccaaaggaatgatgaagctgtgttcggcctttgcctgtcccacgtctaacttacagataattgggctgtggaaatggcagtaccctgtgcaaataaggaagctgctggaggaagtgcagctactcaagccccgagtcgtaattgacggtagttggcattcttttgatgaagatgaccggtactggtggaaaaactgaagatacggaaacctgccccactcacacccatctgatggaggaactttaaacgctgttttctcagagcaagctatgcacctgggagttccttctcaaagatggagaatgatttctgattctcacaaagccctcaatggtagtgattcttctgtgttcactctacgttggttactggatttgaaggctagagaccttcaagtcataggactcagtatctgtgaaatgtccgtcatatctcagagcatatagagggaattaaataaacacaaagcatttggaaaa

SEQ ID NO: 4 - protein, 1200 aa, Homo sapiens
MKVAGGLELGAAALLSASPRALVTLSTGPTCSILPKNPLFPQNLSSQPCIKMEGDKSLTFSSYGLQWCLYELDKEEFQTFKELLKKKSSESTTCSIPQFEIENANVECLALLLHEYYGASLAWATSISIFENMNLRTLSEKARDDMKRHSPEDPEATMTDQGPSKEKVPGISQAVQQDSATAAETKEQEISQAMEQEGATAAETEEQEISQAMEQEGATAAETEEQGHGGDTWDYKSHVMTKFAEEEDVRRSFENTAADWPEMQTLAGAFDSDRWGFRPRTVVLHGKSGIGKSALARRIVLCWAQGGLYQGMFSYVFFLPVREMQRKKESSVTEFISREWPDSQAPVTEIMSRPERLLFIIDGFDDLGSVLNNDTKLCKDWAEKQPPFTLIRSLLRKVLLPESFLIVTVRDVGTEKLKSEVVSPRYLLVRGISGEQRIHLLLERGIGEHQKTQGLRAIINNRELLDQCQVPAVGSLICVALQLQDVVGESVAPFNQTLTGLHAAFAFHQLTPRGVVRRCLNLEERVVLKRFCRMAVEGVWNRKSVFDGDDLMVQGLGESELRALFHMNILLPDSHCEEYYTFFHLSLQDFCAALYYVLEGLEIEPALCPLYVEKTKRSMELKQAGFHIHSLWMKRFLFGLVSEDVRRPLEVLLGCPVPLGVKQKLLHWVSLLGQQPNATTPGDTLDAFHCLFETQDKEFVRLALNSFQEVWLPINQNLDLIASSFCLQHCPYLRKIRVDVKGIFPRDESAEACPVVPLWMRDKTLIEEQWEDFCSMLGTHPHLRQLDLGSSILTERAMKTLCAKLRHPTCKIQTLMFRNAQITPGVQHLWRIVMANRNLRSLNLGGTHLKEEDVRMACEALKHPKCLLESLRLDCCGLTHACYLKISQILTTSPSLKSLSLAGNKVTDQGVTPLSDALRVSQCALQKLILEDCGITATGCQSLASALVSNRSLTHLCLSNNSLGNEGVNLLCRSMRLPHCSLQRLMLNQCHLDTAGCGSLALALMGNSWLTHLSLSMNPVEDNGVKLLCEVMREPSCHLQDLELVKCHLTAACCESLSCVISRSRHLKSLDLTDNALGDGGVAALCEGLKQKNSVLTRLGLKACGLTSDCCEALSLALSCNRHLTSLNLVQNNFSPKGMMKLCSAFACPTSNLQIIGLWKWQYPVQIRKLLEEVQLLKPRVVIDGSWHSFDEDDRYWWKN

SEQ ID NO: 5 - DNA, 24 bp, Artificial
  Synthetic oligonucleotide
ttaagagggtgtctgggggatgtt

SEQ ID NO: 6 - protein, 4 aa, Homo sapiens
DEVD

SEQ ID NO: 7 - protein, 4 aa, Homo sapiens
SHVD

SEQ ID NO: 8 - protein, 4 aa, Homo sapiens
DBLD

SEQ ID NO: 9 - protein, 4 aa, Homo sapiens
DGPD

SEQ ID NO: 10 - protein, 4 aa, Homo sapiens
DEPD

SEQ ID NO: 11 - protein, 4 aa, Homo sapiens
DGTD

SEQ ID NO: 12 - protein, 4 aa, Homo sapiens
DLND

SEQ ID NO: 13 - protein, 4 aa, Homo sapiens
DEED

SEQ ID NO: 14 - protein, 4 aa, Homo sapiens
DSLD

SEQ ID NO: 15 - protein, 4 aa, Homo sapiens
DVPD

SEQ ID NO: 16 - protein, 4 aa, Homo sapiens
WEHD

SEQ ID NO: 17 - protein, 4 aa, Homo sapiens
DEHD

SEQ ID NO: 18 - protein, 4 aa, Homo sapiens
LEHD

SEQ ID NO: 19 - protein, 4 aa, Homo sapiens
VEHD

SEQ ID NO: 20 - protein, 4 aa, Homo sapiens
LETD

SEQ ID NO: 21 - protein, 4 aa, Homo sapiens
LEHD

SEQ ID NO: 22 - protein, 4 aa, Homo sapiens
IEPD

SEQ ID NO: 23 - DNA, 22 bp, Artificial
  Primer sequence
ggaccagaatcctgagctgtgt

SEQ ID NO: 24 - DNA, 21 bp, Artificial
  Primer sequence
gaagcctcaggaaggatggat

SEQ ID NO: 25 - DNA, 24 bp, Artificial
  Primer sequence
atcaagatggaaggagacaaatcg

SEQ ID NO: 26 - DNA, 21 bp, Artificial
  Primer sequence
gtttttccaccagtaccggtc

SEQ ID NO: 27 - DNA, 31 bp, Artificial
  Primer sequence
ggctgctggaattgaggcgtcaacactggcc

SEQ ID NO: 28 - RNA, 21 nt, Artificial
  Synthetic oligonucleotide
aagagaagcuggccugauuau

SEQ ID NO: 29 - RNA, 21 nt, Artificial
  Synthetic oligonucleotide
aaauaaucaggccagcuucuc

SEQ ID NO: 30 - DNA, 22 bp, Artificial
  Primer sequence
gatgagatgaggcaggaactga

SEQ ID NO: 31 - DNA, 22 bp, Artificial
  Primer sequence
caggagaaggcacgcacaagag